(19) United States
(12) Patent Application Publication
(10) Pub. No.: US 2026/0245715 A1
(43) Pub. Date: Aug. 20, 2026

(54) SYSTEMS AND METHODS FOR DYNAMIC CONTROL OF MEDICAL DEVICES BASED ON PATIENT-SPECIFIC OPERATIONAL PARAMETERS

(71) Applicant: Imagine Health Group Inc., Miami, FL (US)

(72) Inventors: Mohan Mahadevan, London (GB); Arshak Navruzyan, Paris (FR); Atabak Nezhadfard, London (GB); Vishnu Dev Amara, London (GB); Vitalii Pruks, London (GB); Arthur Levisalles, London (GB)

(21) Appl. No.: 19/541,651

(22) Filed: Feb. 17, 2026

Related U.S. Application Data
(60) Provisional application No. 63/759,278, filed on Feb. 17, 2025.
(60) Provisional application No. 63/759,280, filed on Feb. 17, 2025.

(51) Int. Cl. G16H 40/63; G16H 40/67

(52) U.S. Cl. CPC G16H 40/63; G16H 40/67

(57) ABSTRACT

Systems and methods are provided for patient-specific, real-time adaptive control of medical devices that physically interact with a patient during execution of a medical procedure. One or more sensing elements acquire patient-specific data, and a computing device generates a structured representation of patient state using multi-stage inference, including estimation of external body configuration, device-patient interaction state, and localization of a patient-specific region of interest. Procedural data acquired within the region of interest is compared to reference representations to determine whether an acceptance criterion is satisfied. Based on this evaluation, the computing device maps the inferred patient state to a bounded operational adaptation space defining permissible modifications of controllable device variables and generates control signals that modify at least one physical operational state of the device. The closed-loop architecture iteratively refines device operation during the procedure, enabling dynamic adaptation across diagnostic, interventional, therapeutic, and assistive modalities.

### RELATED APPLICATIONS

This application claims priority to U.S. Provisional Patent Application Nos. 63/759,278 and 63/759,280, both filed Feb. 17, 2025, the entirety of each of which is incorporated herein by reference.

The present application is related to U.S. Patent Application entitled “SYSTEMS AND METHODS FOR CONTROLLED AUTONOMOUS PHYSICAL INTERACTION IN MEDICAL PROCEDURES,” filed on even date herewith and claiming priority to the same provisional applications identified above. The disclosure of that application is incorporated herein by reference in its entirety for all purposes. The present application and the related application describe complementary but distinct aspects of controlled autonomous physical interaction in medical procedures, and each is intended to define independently patentable subject matter.

### TECHNICAL FIELD

This application relates generally to systems, devices, and computer-implemented methods for patient-specific, real-time adaptive control of diagnostic sensors, imaging systems, robotic manipulators, and interventional medical devices during execution of a medical procedure. The disclosed techniques are applicable to a wide variety of medical procedures and clinical workflows, including diagnostic, interventional, invasive, and non-invasive procedures, and optionally involve imaging, sensing, therapeutic delivery, or procedural execution across diverse anatomical and procedural contexts.

### BACKGROUND

Medical diagnostic and interventional procedures frequently require controlled physical interaction between a medical device and a human body. In many such procedures, device performance, data quality, and clinical outcomes are highly sensitive to patient-specific factors, including anatomical variation, tissue composition, physiological state, spatial configuration, and procedural context. These factors vary not only between patients but also within a single procedure as patient position, device interaction, or physiological conditions change over time. Examples include diagnostic imaging procedures, physiological monitoring, image-guided localization or targeting, non-invasive or minimally invasive therapeutic interventions, and supportive or assistive medical procedures.

In diagnostic imaging procedures such as ultrasound imaging, patient-specific variability introduces substantial complexity into the acquisition process. Image formation depends on physical propagation characteristics—such as acoustic transmission, attenuation, reflection, and scattering—that vary across tissue types and anatomical configurations. Increased adipose tissue thickness attenuates ultrasound energy and reduces signal-to-noise ratio. Variations in thoracic geometry, abdominal depth, organ displacement, or rib spacing alter the effective optimization domain and the distance between the probe and target anatomy, requiring corresponding adjustment of imaging depth, focal zones, gain profiles, frequency selection, and probe orientation. Anatomical variants—including differences in organ size, rotation, or position—further necessitate individualized probe placement and hardware configuration. Even within anatomically typical patients, localized pathology or focal diagnostic targets may require selective modification of acquisition parameters to optimize visibility and diagnostic precision.

Because raw signal quality is determined at the point of physical interaction and data acquisition, post-processing software cannot fully compensate for suboptimal hardware settings or inadequate acoustic coupling. Parameters such as probe pose, applied contact force, acquisition depth, frequency, gain, and focal configuration directly influence the quality and diagnostic value of the acquired data. When these hardware-level parameters are not appropriately configured for a specific patient, the resulting data may lack sufficient signal content, leading to nondiagnostic studies, repeat examinations, or reduced diagnostic reliability.

Despite this inherent variability, many diagnostic and interventional devices are operated using static presets, fixed parameter ranges, or protocol-driven workflows defined in advance of a procedure. Device operation is often selected manually by an operator based on general guidelines, manufacturer-recommended defaults, or population-averaged assumptions. While such approaches simplify device operation, they frequently fail to account for patient-specific anatomical and interaction-dependent variability encountered in clinical practice. As a result, device performance is inconsistent, data quality varies across operators and patients, and clinical outcomes depend heavily on operator experience and subjective judgment.

In many existing systems, adaptation—if present—is limited to manual tuning of isolated parameters or the execution of predefined routines that do not incorporate structured, multi-stage inference of patient-specific state. Conventional approaches generally do not differentiate between external body configuration, device-patient interaction state, and localized anatomical target identification as distinct but interrelated stages of inference. Without staged and structured estimation of patient state, adaptation may occur without sufficient contextual understanding of spatial alignment, target localization, or internal anatomical orientation.

Furthermore, prior systems often lack mechanisms for explicit identification and localization of patient-specific regions of interest during execution of a procedure. In the absence of localized targeting, device optimization may occur globally or heuristically, rather than being constrained to the specific anatomical structure or procedural objective relevant to the examination. This can result in inefficient parameter adjustment, excessive exploration of irrelevant parameter space, or suboptimal acquisition quality within diagnostically significant regions.

Conventional systems also generally do not implement structured comparison between acquired data and reference representations corresponding to desired procedural states or target outcomes. Quality evaluation may occur retrospectively or be limited to threshold-based checks on individual parameters rather than holistic assessment of similarity to canonical or context-conditioned reference representations. Without reference-based evaluation tied to an explicit acceptance criterion, iterative improvement of device operation lacks a principled stopping condition and may remain inconsistent or operator dependent.

Similar limitations arise in other diagnostic and interventional procedures in which device behavior must be adapted dynamically to patient-specific conditions. Adaptation logic is often tightly coupled to specific devices, sensing modalities, or proprietary control implementations, limiting applicability across different clinical workflows, anatomical targets, or device platforms. Extending such systems to new procedures or patient populations frequently requires redesign of device-specific control logic rather than reuse of a generalized adaptive framework.

In regulated clinical environments, these limitations are particularly significant. Medical devices must operate within defined safety and performance constraints while delivering consistent and reliable performance across diverse patient populations. Static presets and population-averaged control strategies are typically insufficient to ensure optimal operation for individual patients, while excessive reliance on manual operator adjustment increases cognitive burden, introduces variability, and reduces auditability of decision processes. There is therefore a need for systems capable of inferring patient-specific state in structured stages, localizing procedural targets within patient-specific coordinate frames, evaluating acquired data relative to reference criteria, and adaptively modifying device behavior in a bounded and auditable manner during execution of a procedure.

Accordingly, there exists a need for a technical solution that provides patient-specific, real-time adaptive control of diagnostic and interventional medical devices. Such a solution should continuously acquire multimodal sensing data during physical interaction, generate structured representations of patient-specific state across multiple inference stages, identify and align localized regions of interest relevant to procedural objectives, evaluate procedural data relative to reference representations or defined acceptance criteria, and iteratively modify hardware-level device operation within constrained operational bounds. The solution should operate during execution of the procedure rather than relying on static presets or preconfigured strategies, and should be applicable across a wide range of non-invasive and minimally invasive diagnostic and interventional contexts independent of specific device vendors, sensing modalities, or control implementations.

### SUMMARY

The present invention overcomes the above-identified challenges of existing systems by providing universal systems and methods for patient-specific, real-time adaptive control of diagnostic and interventional medical devices that physically interact with the human body. The invention establishes a closed-loop control framework in which device operation is dynamically adapted during execution of a medical procedure based on patient-specific anatomical, physiological, spatial, and contextual characteristics inferred from real-time sensing data.

In accordance with the invention, one or more sensors acquire patient-specific data during a procedure. Based on the acquired data, one or more computational processes perform structured inference of patient-specific state. In certain embodiments, such inference occurs in multiple stages, including inference of external body configuration, inference of device-patient interaction state, and identification or refinement of one or more patient-specific regions of interest corresponding to a procedural objective. The inferred state defines a constrained operational adaptation space within which device operation can be modified. In some embodiments, the operational adaptation space is represented as a bounded multidimensional parameter space defining permissible variations of controllable device variables subject to safety, physical, procedural, and contextual constraints.

An adaptive control policy module maps the inferred patient-specific state to a dynamically constrained operational adaptation space defining permissible modifications of electromechanical device behavior, and modifies device operation in real time to achieve one or more procedural objectives while respecting applicable constraints. Adaptation occurs iteratively and continuously, enabling device behavior to evolve as patient conditions, anatomical alignment, or procedural context change during execution. The operational adaptation space can include modification of physical interaction parameters, including contact force, pressure distribution, impedance interaction, or other mechanical interaction characteristics between the device and the patient.

In some embodiments, adaptive control includes comparison of acquired procedural data to one or more reference representations associated with desired target states, anatomical configurations, or protocol-defined outcomes. Similarity or quality evaluation can be performed using one or more quantitative or learned metrics. If an acceptance criterion is satisfied, the procedure progresses; if not, adaptive modification of device operation is invoked. Such modification includes adjustment of device pose, sensing parameters, acquisition geometry, energy delivery, interaction conditions, or other controllable variables within the operational adaptation space. The adaptive process iterates until one or more acceptance criteria are met or supervisory input modifies control authority.

Adaptive control is not limited to static parameter tuning and can include modification of device parameters, motion, trajectory, timing, sensing strategy, region-of-interest alignment, or other operational behaviors. In some embodiments, adaptation of multiple parameters occurs in a staged or ordered sequence, where optimization of one parameter informs or constrains optimization of another. Adaptation can occur continuously or at discrete intervals and may be performed autonomously or semi-autonomously, with optional human supervision or override. The invention does not require any specific inference method or control algorithm and can employ rule-based logic, model-based control, optimization methods, data-driven models, machine learning techniques, or combinations thereof.

The invention further provides a system comprising one or more patient-facing or environment-facing sensors, one or more processing units configured to infer patient-specific state from sensed data, an adaptive control module configured to modify device operation in real time based on the inferred state and optional reference evaluation, and a feedback loop enabling continuous evaluation and iterative adaptation during execution of a procedure. The system includes one or more interfaces for human supervision, confirmation, or override, and can be implemented using local, remote, or distributed computing resources. The system is independent of specific device vendors, sensor modalities, algorithms, anatomical targets, or clinical environments.

In one non-limiting embodiment, the invention is applied to ultrasound imaging systems. In this embodiment, ultrasound probe positioning, acquisition depth, gain or amplification profiles, focal configuration, frequency selection, scanning behavior, or control policies are dynamically adapted in real time within a patient-specific anatomical region of interest (ROI). Adaptive modification includes staged or ordered optimization of imaging parameters based on comparison of acquired data to reference representations or quality metrics. Ultrasound imaging is provided as an illustrative instantiation and does not limit the scope of the invention.

More broadly, the disclosed adaptive control framework is applicable to a wide range of non-invasive and minimally invasive diagnostic and interventional procedures, including imaging, physiological monitoring, image-guided localization or targeting, non-invasive therapeutic or assistive interventions, and other procedures in which device behavior must be adjusted dynamically to individual patient variability. By performing structured patient-state inference and constraining device adaptation within a defined operational adaptation space, the control framework reduces reliance on manual parameter tuning, mitigates operator-dependent variability, improves acquisition efficiency, reduces repeated device repositioning, and enhances consistency of procedural execution across heterogeneous patient populations.

In accordance with the invention, a system is provided for patient-specific, real-time adaptive control of a diagnostic or interventional medical device during execution of a medical procedure. The system implements a closed-loop control architecture that continuously acquires sensing data, performs structured patient-state inference, evaluates procedural data relative to reference criteria, and dynamically adapts device operation in real time.

The system comprises one or more sensing elements configured to acquire patient-specific or environment-specific data during interaction between the device and a human body. The sensing elements can be patient-facing, device-facing, or environment-facing, and can include, but are not limited to, imaging sensors, depth sensors, force or torque sensors, inertial or motion sensors, physiological sensors, environmental sensors, or combinations thereof. In some embodiments, the system detects respiratory phase, breath-hold compliance, or voluntary patient motion using multi-modal sensing data. Acceptance criteria evaluation may be gated or weighted based on detected motion state to prevent optimization during respiratory artifacts. It should be appreciated that the number, type, placement, and modality of sensors are not limiting, and sensing elements can be integrated with the device, mounted on an end effector, disposed on a robotic or actuated mechanism, or positioned external to the device in the patient environment.

The system further comprises one or more processing units configured to receive and process the acquired sensing data and to perform multi-stage inference of patient-specific state. In some embodiments, the processing units include modules configured to estimate external body configuration, spatial alignment, or surface landmarks; modules configured to estimate device-patient interaction state or internal anatomical orientation; and modules configured to identify or refine one or more patient-specific regions of interest associated with a procedural objective. The inferred state representation provides contextual information for adaptive control.

The system can further comprise a reference repository configured to store one or more reference representations corresponding to desired procedural targets, anatomical configurations, protocol-defined states, or quality exemplars. The reference repository can be local or remote and can be indexed by anatomical target, protocol identifier, patient characteristics, operator input, or other contextual information. In some embodiments, the processing units retrieve reference representations during execution of a procedure.

In some embodiments, the system further includes a similarity or evaluation module configured to compare acquired procedural data, including data localized within a region of interest, to one or more retrieved reference representations. The evaluation module computes one or more similarity, quality, or consistency measures using structural, statistical, feature-based, learned, or composite metrics. Based on such evaluation, the system can determine whether an acceptance criterion has been satisfied.

An adaptive control module is operatively coupled to the processing units and evaluation module and is configured to modify device operation in real time based on the inferred patient-specific state and evaluation results. The adaptive control module maps inferred patient states to an operational adaptation space defining controllable aspects of device behavior. Such controllable aspects include, but are not limited to, device parameters, control policies, motion trajectories, interaction forces, sensing strategies, acquisition geometry, timing, energy delivery, or other operational characteristics.

If an evaluation module determines that an acceptance criterion has been satisfied, the adaptive control module maintains current operation or progress to a subsequent stage of a procedural protocol. If an acceptance criterion is not satisfied, the adaptive control module initiates iterative modification within the operational adaptation space. Such modification involves incremental or staged adjustment of device pose, sensing configuration, acquisition parameters, or other variables.

In some embodiments, the adaptive control module can implement staged or ordered optimization of multiple parameters. Optimization of one parameter can precede, inform, or constrain optimization of another. The ordering can be predefined, protocol-driven, dynamically determined, or influenced by human supervisory input.

The system further includes a feedback loop enabling continuous real-time adaptation of device operation. Through the feedback loop, sensing data acquired during execution of the procedure is repeatedly evaluated, patient-specific states are updated, regions of interest (ROIs) are refined, reference comparisons are repeated, and device adaptations are iteratively adjusted. Each adaptive iteration results in a modification of at least one physical or machine-operational state of the device, including actuator position, sensing configuration, control gain, trajectory profile, energy emission parameters, or other hardware-level control variables. This closed-loop architecture allows device behavior to evolve dynamically as patient conditions, anatomical alignment, or procedural context change, without requiring interruption or restart of the procedure.

In some embodiments, the system further comprises a safety and constraint enforcement component configured to ensure that adaptive device operation remains within defined operational limits. The safety and constraint enforcement component bounds motion ranges, interaction forces, energy delivery parameters, acquisition settings, and other controllable operational characteristics within predefined safety envelopes. The safety and constraint enforcement component can operate continuously and independently of higher-level adaptive logic, and may approve, modify, constrain, or reject proposed adaptive modifications prior to execution. Constraint enforcement ensures that dynamic adaptation occurs within clinically acceptable limits while preserving responsiveness to patient-specific variability.

In some embodiments, the system further includes one or more human interfaces enabling supervision, confirmation, modification, or override of adaptive device operation. A human operator may be located locally or remotely and may monitor procedural state, review inferred patient-state representations, adjust acceptance criteria, influence region-of-interest selection, modify protocol progression, or alter adaptation strategy. Human supervision may operate concurrently with autonomous adaptation and does not limit the system's ability to perform real-time adaptive control within the defined operational adaptation space.

It should be appreciated that, in some embodiments, the disclosed system is not limited to advisory or decision-support functionality. In these embodiments, the system is configured to condition (e.g., generate, constrain, filter, modify, and/or reject) control signals generated from human input prior to execution by the robotic device and/or the procedural device. Human-generated control inputs, including teleoperated commands, are routed through the adaptive control and safety monitoring layers and are subject to constraint within the defined operational adaptation space. Accordingly, even in shared-control or teleoperated embodiments, physical device state transitions occur only within system-defined bounds derived from patient-specific inference and acceptance-based evaluation. The system therefore maintains architectural control over permissible electromechanical transformations, regardless of whether final actuation is initiated autonomously or through human interaction.

In some embodiments, the disclosed adaptive control processes are implemented using one or more processors configured to execute stored instructions that transform incoming sensor signals into structured patient-state representations and corresponding control commands that modify operation of electromechanical, imaging, or energy-delivery subsystems of the device. It should be appreciated that the systems and methods described herein are independent of any specific device vendor, sensor modality, control algorithm, anatomical target, or clinical environment, and are applicable to a wide range of non-invasive and minimally invasive diagnostic and interventional procedures.

The invention, in one aspect, features a system for patient-specific adaptive control of a medical device configured to physically interact with a patient during execution of a medical procedure. The system includes one or more sensing elements configured to acquire sensing data during interaction between the medical device and the patient. The system also includes a computing device operatively coupled to the one or more sensing elements. The computing device generates a structured representation of patient-specific state based on the sensing data. The computing device identifies a patient-specific region of interest associated with a procedural objective. The computing device compares procedural data acquired within the region of interest to one or more reference representations and determines whether an acceptance criterion is satisfied. The computing device maps the structured representation of patient-specific state to a bounded operational adaptation space defining permissible modifications of controllable device variables. The computing device generates control signals that modify at least one physical operational state of the medical device within the bounded operational adaptation space in response to determining whether the acceptance criterion is satisfied, where the medical device comprises one or more actuators configured to execute the generated control signals.

The invention, in another aspect, features a method of patient-specific adaptive control of a medical device configured to physically interact with a patient during execution of a medical procedure. One or more sensing elements acquire sensing data during interaction between the medical device and the patient. A computing device operatively coupled to the one or more sensing elements generates a structured representation of patient-specific state based on the sensing data. The computing device identifies a patient-specific region of interest associated with a procedural objective. The computing device compares procedural data acquired within the region of interest to one or more reference representations and determines whether an acceptance criterion is satisfied. The computing device maps the structured representation of patient-specific state to a bounded operational adaptation space defining permissible modifications of controllable device variables. The computing device generates control signals that modify at least one physical operational state of the medical device within the bounded operational adaptation space in response to determining whether the acceptance criterion is satisfied, where the medical device comprises one or more actuators configured to execute the generated control signals.

Any of the above aspects can include one or more of the following features. In some embodiments, the structured representation of patient-specific state is generated using multi-stage inference comprising: (i) estimation of external body configuration or surface landmarks; (ii) estimation of device-patient interaction state; and (iii) identification or refinement of the patient-specific region of interest. In some embodiments, estimation of the device-patient interaction state comprises determining at least one of: spatial alignment between the medical device and internal anatomy, contact force distribution, impedance characteristics, tissue compliance, acoustic coupling quality, or a coordinate transform between device space and anatomical space.

In some embodiments, a reference repository is operatively coupled to the computing device, the reference repository storing one or more reference representations corresponding to desired anatomical configurations, procedural targets, protocol-defined states, or quality exemplars, and the computing device retrieves the one or more reference representations based on at least one of an inferred patient-state stage, procedural phase, anatomical target, or identified region of interest. In some embodiments, the reference representations comprise at least one of geometric templates, anatomical atlases, statistical models, learned feature embeddings, signal profiles, or canonical parameter sets derived from prior procedures.

In some embodiments, the bounded operational adaptation space comprises a multidimensional parameter space defining permissible ranges of at least one controllable device variable selected from: device pose, motion trajectory, actuator force, contact pressure, compliance parameter, acquisition depth, focal configuration, gain setting, scanning trajectory, timing sequence, or energy delivery parameter. In some embodiments, mapping to the bounded operational adaptation space further comprises dynamically constraining permissible modifications based on at least one of inferred anatomical context, procedural protocol constraints, safety limits, device capability limits, or human supervisory input.

In some embodiments, generation of control signals comprises staged or ordered optimization of multiple controllable device variables. In some embodiments, the computing device independently evaluates proposed modifications within the bounded operational adaptation space against predefined or dynamically determined safety envelopes, and restrict, modify, or override the generated control signals prior to execution by the medical device. In some embodiments, the computing device, the one or more sensing elements, and the medical device are configured in a closed-loop architecture in which (i) execution of the generated control signals modifies the at least one physical operational state of the medical device, (ii) the modified physical operational state of the medical device produces updated sensing data, and (iii) the structured representation of patient-specific state is iteratively updated based on the updated sensing data.

In some embodiments, the computing device is further configured to receive human-generated control inputs corresponding to desired operation of the medical device, and to condition execution of the human-generated control inputs such that any resulting actuation of the medical device remains within the bounded operational adaptation space. In some embodiments, generation of control signals comprises executing an ordered optimization sequence including: (i) first optimizing a mechanical interaction force between the medical device and the patient to satisfy a coupling threshold; and (ii) subsequently optimizing at least one signal acquisition parameter within the patient-specific region of interest after the coupling threshold is satisfied.

In some embodiments, the computing device is further configured to execute a structured perturbation routine in which the medical device performs a programmed sequence of incremental physical movements or parameter adjustments to generate sensing data for refining the structured representation of patient-specific state. In some embodiments, the computing device is further configured to perform a dynamic coordinate transform between a device-centric coordinate system and an anatomical-centric coordinate system defined by an inferred three-dimensional body mesh and one or more identified anatomical landmarks.

### BRIEF DESCRIPTION OF THE DRAWINGS

The advantages of the invention described above, together with further advantages, may be better understood by referring to the following description taken in conjunction with the accompanying drawings. The drawings are not necessarily to scale, emphasis instead generally being placed upon illustrating the principles of the invention.

FIG. 1 is a schematic diagram illustrating an example system configuration for patient-specific, real-time adaptive control of a diagnostic or interventional medical device.

FIG. 2 is a detailed block diagram of the computing device of FIG. 1.

FIG. 3 is a flow diagram of a first stage of patient-state inference in which external body configuration, surface landmarks, pose, or spatial orientation are estimated using sensing data to establish an initial spatial reference for adaptive control.

FIG. 4 is a flow diagram of a second stage of patient-state inference in which device-patient interaction data is used to estimate internal anatomical orientation, spatial alignment between device and anatomy, or patient-specific interaction state relevant to a procedural objective.

FIG. 5 is a diagram of identification and refinement of a patient-specific region of interest corresponding to an anatomical structure or procedural target, and alignment of device operation within the localized region.

FIG. 6 is a diagram of mapping of inferred patient state and region of interest information to an operational adaptation space defining controllable device parameters, motion, or acquisition behaviors subject to procedural and constraint boundaries.

FIG. 7 is a flow diagram of a method for comparing acquired procedural data within a region of interest to reference representations using similarity or quality evaluation metrics to determine whether an acceptance criterion is satisfied.

FIG. 8 is a flow diagram of a method for determining satisfaction of an acceptance criterion based on a comparison between acquired data and reference representations.

FIG. 9 is a flow diagram of a method of staged or ordered adaptive optimization of ultrasound device parameters within the operational adaptation space.

FIG. 10 is a diagram illustrating deterministic geometric estimation of an acoustic window using two-dimensional (2D) and three-dimensional (3D) imaging data.

FIG. 11 is a diagram illustrating determination of an optimal robot/probe location and orientation for an anchor view.

FIG. 12 is a diagram illustrating a reference optimal probe pose and controlled perturbations of the probe pose.

### DETAILED DESCRIPTION

In the following description, embodiments of the disclosed system and methods are described with reference to the accompanying drawings, which illustrate representative implementations of the invention. Specific terminology is used for clarity and descriptive purposes; however, the disclosure is not intended to be limited to the particular terms or examples described. Rather, each described component, feature, or operation is intended to encompass technical equivalents that perform similar functions or achieve similar results.

The following description sets forth exemplary embodiments of systems and methods for patient-specific, real-time adaptive control of diagnostic and interventional medical devices. The embodiments described herein are illustrative and non-limiting, and the invention is not restricted to the specific configurations, components, algorithms, metrics, or sequences disclosed.

As used herein, adaptive control refers to constrained modification of one or more controllable device variables during execution of a medical procedure based on patient-specific information inferred from real-time sensing data and optionally evaluated relative to one or more reference representations. Such modification can include alteration of actuator position, motion trajectory, sensing configuration, energy delivery parameters, signal acquisition characteristics, or other machine-operational variables that govern physical interaction between the device and the patient. The disclosed systems and methods implement a closed-loop control architecture in which sensing, structured inference, region-of-interest identification, evaluation, adaptation, execution, and feedback occur iteratively during interaction between a device and a human body. In some embodiments, the closed-loop control architecture is implemented as a layered system architecture comprising a sensing layer, a patient-state inference layer, an evaluation layer, an adaptive control layer, and an execution layer, each operatively coupled through one or more processing units. The adaptive control framework continuously transforms device operational state in response to updated patient-state representations, enabling device behavior to evolve dynamically as patient conditions or procedural context change, in contrast to conventional systems which typically require manual adjustment, pre-defined protocol sequencing, or parameter presets that do not account for intra-procedural variability or individual anatomical differences.

### System Configuration

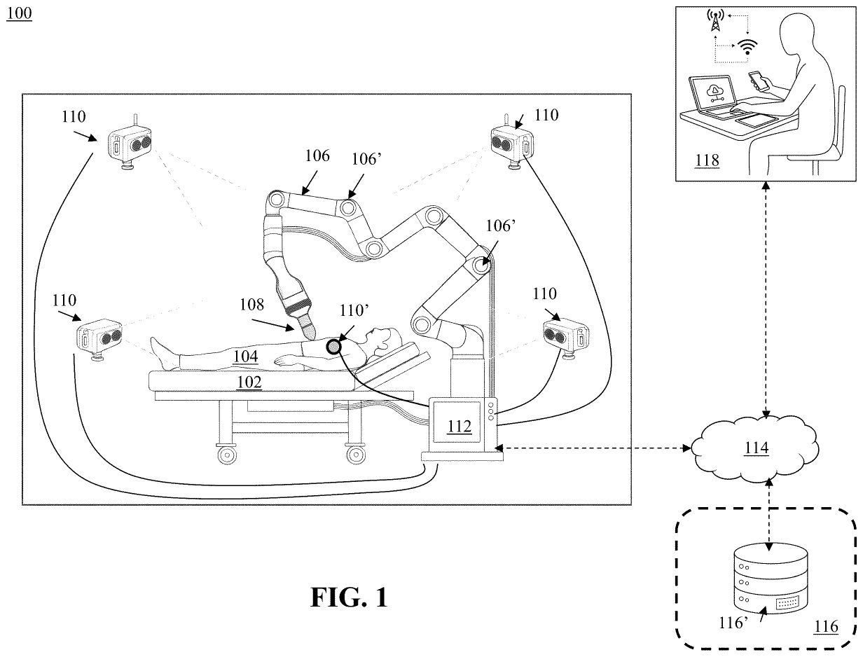

FIG. 1 is a schematic diagram illustrating an example system configuration 100 for patient-specific, real-time adaptive control of a diagnostic or interventional medical device. The system configuration 100 includes a patient support structure 102 (e.g., a bed, table, or other apparatus) for supporting a patient 104 during the medical procedure, a robotic device 106, one or more procedural devices 108 (which are typically affixed to the robotic device 106) configured to perform functions pertaining to execution of the medical procedure on the patient, one or more environment sensors 110 arranged to view and capture data pertaining to the patient, the robot, and/or the surrounding clinical environment, a computing device 112 coupled to the robotic device 106 (and optionally the patient support structure 102), the procedural devices 108, the environment sensors 110, and a communications network 114 connecting the computing device 112 to one or more external computing resources, such as a cloud computing environment 116 including one or more data stores (e.g., database 116′) and a remote computing device 118 operated by a human expert.

The robotic device 106 can comprise any actuated mechanical system configured to position, orient, move, or otherwise manipulate one or more procedural devices 108 (e.g., medical devices, tools, or end effectors) relative to the patient support structure 102 and/or the patient 104. In some embodiments, the robotic device 106 includes one or more articulated joints, links, linear or rotary actuators, or compliant elements providing one or more degrees of freedom, and can be configured to execute commanded motions, trajectories, forces, or interaction states under control of computing device 112. For example, each joint of the robotic device 106 can include, be coupled to, or be associated with one or more sensing elements 106′ configured to measure or estimate joint state and interaction parameters. Such sensing elements include, but are not limited to, position or displacement sensors, rotary or linear encoders, velocity or speed sensors, accelerometers, gyroscopes, inertial measurement units, force or torque sensors, strain or load sensors, compliance or deflection sensors, temperature sensors, or combinations thereof. In some embodiments, joint parameters are measured directly, while in other embodiments one or more parameters are inferred or estimated using actuator signals, dynamic models, or state observers. It should be appreciated that any type, number, placement, and/or modality of joint-level sensing elements can be incorporated into the system 100 without limiting the scope of the technology described herein.

The robotic device 106 can be controlled at one or more abstraction levels, including joint-level control, task-space control, impedance or admittance control, force or torque control, or higher-level interaction objectives. It should be appreciated that control commands received at the robotic device 106 from the computing device 112 can specify desired positions, velocities, forces, compliance parameters, or interaction constraints—rather than explicit joint motions. The robotic device 106 is not limited to a particular form factor and comprises, by way of example and without limitation: an articulated robotic arm, a serial or parallel manipulator, a gantry-based system, a mobile robotic platform, a wearable or body-mounted actuator, or a combination thereof. The robotic device 106 can be fixed, movable, or repositionable relative to the patient support structure 102, the patient 104, and/or the clinical environment.

As mentioned above, the robotic device 106 can also include one or more procedural devices 108 that are configured to physically interact with the patient 104. In the example robotic device 106 of FIG. 1, the procedural devices 108 are positioned at one end of the robotic device 106 closest to the patient 104. Procedural devices 108 can comprise any diagnostic, interventional, or therapeutic apparatus configured to be mechanically coupled to, supported by, or manipulated by the robotic device 106 for performing physical interaction with the patient 104. In some embodiments, the procedural device 108 can be affixed to an end effector of a robotic arm or otherwise operatively coupled to the robotic device, and the device 108 is configured to acquire diagnostic data, perform an intervention, deliver a therapeutic effect, or combinations thereof. Also, it should be appreciated that the procedural device 108 can be rigidly mounted, removably attached, or dynamically coupled to the robotic device 106, and include mechanical, electrical, optical, fluidic, or wireless interfaces. In some embodiments, the procedural device 108 is interchangeable, allowing different procedural devices to be mounted to the same robotic platform without modification of the robotic device 106 itself.

Diagnostic procedural devices can include, but are not limited to, imaging probes, physiological sensing devices, localization or mapping instruments, or other devices configured to acquire diagnostic data through physical interaction with a patient. Interventional procedural devices include devices configured to penetrate, traverse, manipulate, or otherwise interact with tissue, including needles, catheters, cannulas, guidewires, biopsy tools, or similar instruments. Therapeutic procedural devices include devices configured to deliver energy, substances, or mechanical effects to tissue for treatment or modulation of a physiological condition. In some embodiments, a procedural device 108 performs multiple functions, such as acquiring diagnostic data while performing an intervention or delivering therapy, and transitioning between diagnostic, interventional, and therapeutic operation during execution of a medical procedure. Also, the procedural device 108 can be configured to engage in physical interaction with a patient involving contact force, pressure, penetration depth, orientation, motion trajectory, or applied energy, and such interaction parameters are monitored, controlled, or constrained by the controls and/or governance implemented by the computing device 112 and/or the remote device 118 of the human expert.

As described above, the system 100 also includes one or more environment sensors 110. As used herein, environment sensors 110 can comprise one or more sensing elements positioned in, on, or around a clinical environment and configured to acquire data relating to the patient support structure 102, the patient 104, the robotic device 106, the one or more procedural devices 108, human operators (e.g., technicians or other staff that may be present in the clinical environment), and/or environmental conditions (e.g., sound, temperature) during execution of a medical procedure. It should be appreciated that environment sensors 110 can be fixed, movable, or reconfigurable, and can be positioned on walls, ceilings, floors, carts, stands, booms, furniture, wearable mounts, or other structures within the clinical environment. In some embodiments, one or more environment sensors 110 can be integrated into existing clinical infrastructure.

Environment sensors 110 can comprise any of a number of different types, function sets, form factors, or modalities—such as visual sensors, audio sensors, environmental condition sensors, spatial/motion sensors, or presence/identification sensors—depending upon the requirements of the system 100, the medical procedure and/or the clinical environment. Visual environment sensors can include cameras or imaging devices configured to capture images or video of the patient support structure 102, the patient 104, the robotic device 106, the procedural devices 108, or other aspects of the clinical workspace. Audio environment sensors can include microphones or acoustic sensors configured to acquire sound data relating to speech, alarms, mechanical operation, or patient vocalizations. Environmental condition sensors can be configured to measure ambient conditions within the clinical environment, including temperature, humidity, lighting, or air quality. Spatial environment sensors can include range, proximity, or motion sensors configured to detect relative positions or movement of objects, people, or devices within the clinical environment. Presence or identification sensors can be used to detect or identify patients, clinicians, or other personnel within the clinical environment.

In some embodiments, one or more of the environment sensors 110 comprise physiological sensing elements such as electrocardiogram (ECG) leads (110′) configured to acquire electrical activity of the patient's heart during execution of the medical procedure. The ECG leads 110′ may be positioned on the patient's body and operatively coupled to the computing device 112 directly (or via a monitoring system). Signals acquired from the ECG leads 110′ can be sampled, digitized, and transmitted to the computing device 112 for processing in real time. The computing device 112 may use information derived from the ECG signal data—such as heart rate, cardiac cycle phase, rhythm irregularities, or detected trigger events—to inform or adjust the context of the medical procedure, modify operational parameters of the procedural device 108, or synchronize data acquisition to specific phases of the cardiac rhythm.

In some embodiments, data acquired from environment sensors 110 can be used to determine procedural context, monitor safety conditions, detect unexpected events, support authorization decisions, or modify or constrain autonomous physical interaction in real time. Also, in some embodiments, data from environment sensors 110 can be aggregated, fused, or integrated with data from patient support structure-mounted sensors, robotic device-mounted sensors (e.g., procedural sensors 108), joint-level sensors (e.g., 106′), and/or patient-mounted sensors to generate a unified representation of procedural state. In some embodiments, environmental parameters can be estimated or inferred from environmental sensor data using computational models rather than measured directly.

Environment sensors 110 are operatively coupled to computing device 112 for communication of acquired data. Such coupling can be implemented using wired or wireless communication links, including direct connections, networked connections, or combinations thereof. In some embodiments, environment sensors 110 transmit raw sensor data, processed sensor data, metadata, or event signals to the computing device 112 for use in execution of the medical procedure and governance of autonomous physical interaction. Communication can occur continuously, periodically, on demand, or in response to detected events, and can utilize any suitable communication protocol, interface, or transport mechanism.

Computing device 112 includes specialized hardware and/or software modules that execute on one or more processors and interact with memory modules of computing device 112, to receive data from other components of system 100, transmit data to other components of system 100, and perform functions including but not limited to, data acquisition, data processing, data transmission, command generation, and other tasks relating to execution of a medical procedure. The computing device 112 receives data indicative of physical interaction between one or more devices (e.g., procedural devices 108) and a patient, procedural context, environmental conditions (e.g., from environment sensors 110), system state, or patient state, and the computing device 112 can use such data to support execution, monitoring, and governance of autonomous and semi-autonomous physical interaction. As mentioned above, the computing device 112 can be communicatively and/or operatively coupled to the patient support structure 102, the robotic device 106, the procedural devices 108, and the environment sensors 110 for the purpose of acquiring, processing, transmitting, and managing data associated with execution of a medical procedure. Generally, computing device 112 is configured to execute one or more software modules to perform its designated functions. In some embodiments, the computing device 112 can be implemented as a single computing unit (e.g., located in the clinical environment) or as a distributed computing architecture comprising multiple computing nodes, controllers, or processing modules. Although FIG. 1 depicts a single computing device 112 in the clinical environment, it should be appreciated that any number of computing devices, arranged in a variety of architectures, resources, and configurations (e.g., cluster computing, virtual computing, cloud computing) can be used without departing from the scope of the technology described herein.

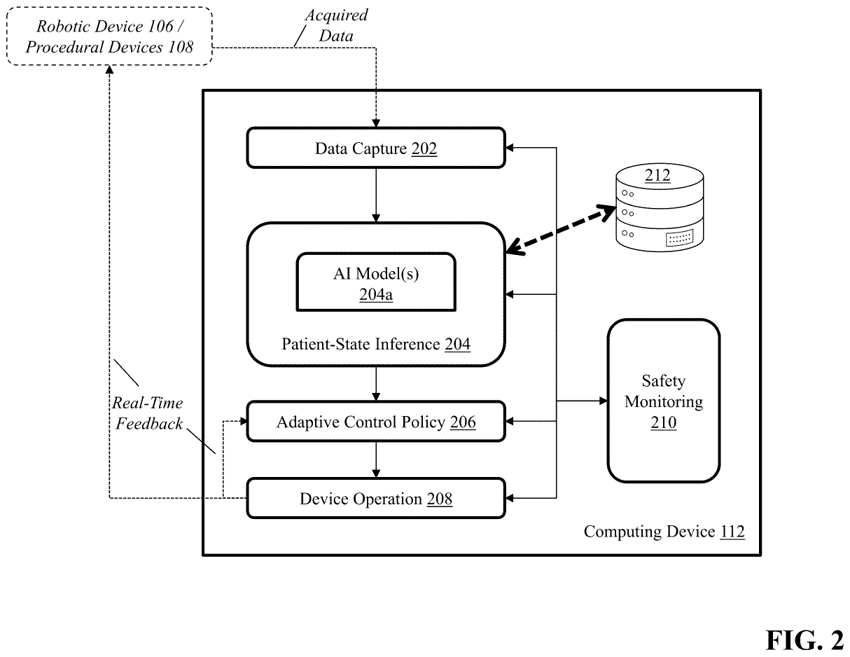

FIG. 2 is a detailed block diagram of the computing device 112 of FIG. 1. As shown in FIG. 1, the computing device 112 includes a data capture module 202, a patient-state inference module 204 with one or more AI models 204a, an adaptive control policy module 206, a device operation module 208, a safety monitoring module 210, and a reference repository 212. In some embodiments, modules 202, 204, 206, 208, 210, and 212 are specialized sets of computer software instructions programmed onto one or more dedicated processors in computing device 112 and can include specifically designated memory locations and/or registers for executing the specialized computer software instructions.

Although modules 202, 204, 206, 208, 210, and 212 are shown in FIG. 2 as executing within a single computing device 112, in some embodiments the functionality of modules 202, 204, 206, 208, 210, and 212 can be distributed among a plurality of computing devices (including, but not limited to, one or more computing resources in cloud computing environment 116). Computing device 112 enables modules 202, 204, 206, 208, 210, and 212 to communicate with each other in order to exchange data for the purpose of performing the described functions.

In some embodiments, data capture module 202 is configured to acquire data from other devices/components of system 100 relating to execution of a medical procedure. The data captured by module 202 includes, but is not limited to, data from patient support structure 102, robotic device 106, robotic device sensors 106′, procedural devices 108, environment sensors 110 and/or other user input devices. Such data includes images, video, audio signals, force or torque measurements, device pose or motion data, physiological signals, environmental measurements, timestamps, and metadata. The data capture module 202 can operate continuously, periodically, or be event-driven, and the module 202 can perform functions such as time synchronization, buffering, preprocessing, or formatting of acquired data for downstream processing.

In some embodiments, patient state-inference module 204 is configured to receive acquired data from data capture module 202 and generate one or more structured representations of patient-specific state during execution of a medical procedure. The patient state-inference module 204 can perform one or more computational operations including filtering, normalization, feature extraction, sensor fusion, state estimation, and transformation of raw sensor data into representations suitable for analysis or decision support. The patient state-inference module 204 can operate in real time or near real time, and module 204 generates intermediate outputs used by AI models (e.g., AI model(s) 204a), adaptive control logic, or safety monitoring components.

Patient-state inference module 204 can generate multi-level or staged representations of patient-specific state. In some embodiments, inference may occur in multiple stages, including: (i) estimation of external body configuration or anatomical surface topology; (ii) estimation of device-patient interaction state, including contact condition, force distribution, impedance characteristics, or relative alignment; and (iii) identification, localization, or refinement of one or more patient-specific regions of interest (ROIs) associated with a procedural objective. In some embodiments, patient-state inference module 204 can further estimate procedural phase, anatomical target classification, or task-specific context.

In some embodiments, the patient-state inference module 204 further determines a procedural phase corresponding to a standardized clinical protocol comprising multiple required imaging views. The adaptive control framework dynamically adjusts region-of-interest localization, acceptance criteria, and operational adaptation space constraints based on the currently active protocol-defined view (e.g., parasternal long axis, parasternal short axis, apical, subcostal, suprasternal notch). Completion of required views may be tracked and verified prior to procedural progression.

The patient-state inference module 204 can include one or more computational models, including AI model(s) 204a, rule-based systems, statistical estimators, dynamic state observers, geometric reconstruction modules, or combinations thereof. The AI models 204a are configured to perform a number of different data analysis tasks including, without limitation, perception, procedural state estimation, prediction, quality assessment, anomaly detection, or generation of candidate actions or recommendations. The AI model 204a can receive inputs such as sensor-derived features, device state information, protocol parameters, or contextual data that originate at one or more other components of system 100, and the model 204a can produce outputs including inferred patient-specific procedural states, estimated parameters, confidence measures, quality scores, or suggested next procedural steps. The AI model 204a generates outputs that can be provided to patient-state inference module 204, adaptive control policy module 206, and/or safety monitoring module 210 for further processing. It should be appreciated that in a typical embodiment, the output from AI model 204a is not used by itself to authorize physical interaction with a patient.

In some embodiments, the AI model(s) 204a and/or other computational models described herein are trained using supervised, semi-supervised, self-supervised, reinforcement learning, or hybrid learning approaches based on data stored in reference repository 212 or other curated datasets. During runtime, trained models generate predictions, inferred state representations, similarity measures, confidence estimates, or recommended adaptations in response to real-time sensing data. No particular training methodology, loss function, model architecture, or optimization strategy is required, and alternative model forms may be employed without departing from the scope of the disclosed adaptive control framework.

Patient-state inference module 204 is coupled to reference repository 212, which may be implemented as one or more local or remote data stores (e.g., located in cloud computing environment 116) operatively coupled to computing device 112. Reference repository 212 stores one or more reference representations corresponding to desired anatomical configurations, protocol-defined states, quality exemplars, procedural targets, statistical models, or learned representations derived from prior procedures or training data. Reference representations can include, but are not limited to, geometric templates, anatomical atlases, signal profiles, image exemplars, target parameter ranges, procedural phase definitions, safety envelopes, optimization objectives, or composite feature vectors. Reference data stored in the reference repository 212 can be retrieved by the patient state-inference module 204 during execution of a medical procedure, as described herein.

In some embodiments, the reference repository 212 further comprises a reference database storing synchronized multi-modal sensing data acquired during prior procedures. Stored data may include raw sensing data, processed feature representations, inferred patient-state parameters, region-of-interest annotations, device adaptation histories, acceptance-criterion evaluations, and associated procedural outcome measures. The reference database therefore contains structured examples of patient-specific variability across anatomical, mechanical, physiological, and contextual dimensions.

Data stored in the reference database can be collected using expert-guided procedures, structured perturbation-based exploration, staged acquisition sequences, or other systematic data collection techniques designed to capture clinically relevant variability while respecting operational and safety constraints. In some embodiments, perturbation-based exploration can intentionally vary device pose, contact conditions, acquisition parameters, or energy delivery settings within bounded safety envelopes to generate diverse training and reference examples.

Reference representations retrieved during execution of a procedure may be derived from such stored datasets using statistical aggregation, clustering, similarity indexing, learned embeddings, or other transformation techniques. In some embodiments, the reference repository 212 supports retrieval of canonical anatomical templates, representative parameter sets, probabilistic distributions, or patient-specific exemplars derived from historical procedure data.

In some embodiments, retrieval of reference representations from reference repository 212 by patient state-inference module 204 is context-conditioned and stage-specific. The patient state-inference module 204 can select reference data based on the current inferred patient-state stage, procedural phase, anatomical target, device-patient interaction state, or identified region of interest. For example, reference representations associated with an initial anatomical localization stage can differ from reference representations associated with parameter optimization within a localized region. Accordingly, reference repository 212 may support dynamic selection of stage-appropriate reference data as the patient-state inference module 204 refines its structured representation during execution of the procedure.

In some embodiments, the AI model(s) 204a and other associated computational models described herein are developed, validated, deployed, and updated within a structured model training and lifecycle architecture that is logically distinct from the real-time procedural control architecture. The model training architecture operates in an offline, cloud-based, distributed, simulated, or hybrid computing environment and is configured to generate trained model parameters used during runtime inference within computing device 112. Separation of training and runtime environments ensures that model development, validation, and performance optimization occur independently of live procedural execution.

Training data used to develop AI model(s) 204a can be derived from data stored in the reference repository 212 and/or one or more external data sources. Such training data can include synchronized multi-modal sensing data acquired during prior procedures, including imaging data, force and torque measurements, pose information, physiological signals, environmental context data, and associated procedural metadata. In some embodiments, training datasets further include structured annotations, ground-truth labels, anatomical segmentation maps, device-alignment indicators, procedural phase markers, acceptance-criterion outcomes, and post-procedural outcome measures. Labels may be generated by expert annotation, semi-automated tools, consensus review, simulation-derived ground truth, or statistical aggregation of multiple observations.

In some embodiments, training data is augmented through structured perturbation-based exploration, simulation environments, digital anatomical models, or physics-based tissue interaction models. Controlled variations of probe pose, contact force, scanning trajectory, acquisition depth, focal configuration, or other device parameters within bounded safety envelopes may be performed to generate diverse training examples. Synthetic data may be generated using computational anatomical atlases, deformable body models, ray-tracing simulations, acoustic propagation models, or biomechanical simulations to supplement real-world procedural datasets and improve generalization across patient populations.

Feature extraction pipelines are applied to training data prior to model optimization. Such pipelines may include filtering, normalization, time-window aggregation, spatial registration, coordinate transformation, dimensionality reduction, feature embedding generation, and multi-modal sensor fusion. Features may include geometric descriptors, texture descriptors, signal frequency components, force-displacement gradients, anatomical alignment transforms, motion trajectories, compliance estimates, probabilistic confidence measures, or composite feature vectors. In some embodiments, learned embeddings generated by intermediate neural network layers are stored as structured representations within reference repository 212 for subsequent similarity indexing or clustering.

Model training may employ supervised learning, semi-supervised learning, self-supervised learning, unsupervised representation learning, reinforcement learning, imitation learning, probabilistic graphical modeling, optimization-based fitting, or hybrid approaches. Loss functions may incorporate structural similarity measures, alignment error minimization, probabilistic likelihood maximization, safety-bound adherence penalties, convergence criteria, or composite objective functions reflecting both procedural performance and safety constraints. In reinforcement learning embodiments, reward signals may incorporate anatomical alignment quality, signal clarity metrics, procedural efficiency measures, and safety-bound compliance.

Following training, model parameters are subjected to validation procedures prior to deployment. Validation may include cross-validation against held-out datasets, stratified performance analysis across demographic or anatomical subgroups, robustness testing under simulated perturbations, safety-bound stress testing, bias analysis, uncertainty quantification, and comparison to predefined benchmark criteria. In some embodiments, a model deployment gate requires satisfaction of predefined performance thresholds before model parameters are authorized for integration into runtime inference module 204.

Trained models may be versioned and stored within reference repository 212 or external model registries, together with metadata identifying training dataset composition, feature definitions, training hyperparameters, validation performance metrics, and deployment authorization status. Model version control supports auditability, rollback capability, and regulatory traceability. In some embodiments, computing device 112 retrieves authorized model versions from cloud computing environment 116 subject to authentication and integrity verification procedures.

During runtime execution of a medical procedure, AI model(s) 204a operate strictly in inference mode. Runtime inference transforms incoming real-time sensing data into structured patient-state representations, similarity metrics, confidence measures, or candidate adaptation suggestions. In some embodiments, inference outputs are accompanied by uncertainty estimates, probability distributions, or confidence intervals reflecting model certainty. Outputs generated by AI model(s) 204a are not directly transmitted to device operation module 208. Instead, inference outputs are first processed by patient-state inference module 204, combined with rule-based logic, procedural constraints, and reference evaluations, and subjected to safety monitoring by safety monitoring module 210 prior to authorization of any physical device modification.

In some embodiments, online adaptation or limited incremental learning may occur during runtime, provided that such adaptation remains bounded within predefined parameter limits and does not modify core model weights without authorization. For example, adaptation may include updating normalization statistics, refining patient-specific embedding representations, adjusting similarity thresholds, or tuning search bounds within the operational adaptation space. Any modification of primary model parameters may require offline retraining and revalidation consistent with the model lifecycle procedures described above.

The model lifecycle architecture may further include mechanisms for performance monitoring and drift detection. Runtime performance metrics, acceptance-criterion satisfaction rates, anomaly detection signals, and outcome measures may be logged and periodically analyzed to detect distributional shift, degradation in model performance, or emergence of bias. If drift is detected, retraining may be initiated using updated datasets while maintaining separation between training and live procedural execution environments.

In some embodiments, federated learning architectures may be employed in which distributed clinical sites perform local model updates using site-specific data while sharing aggregated, privacy-preserving model parameter updates with a centralized training environment. Such architectures may utilize secure aggregation protocols, differential privacy mechanisms, or encryption techniques to preserve patient confidentiality while enabling model improvement across diverse patient populations.

The structured model training and lifecycle architecture described herein ensures that artificial intelligence components remain auditable, updatable, and constrained within defined safety and performance envelopes. By separating model development from runtime procedural control, enforcing validation gates prior to deployment, and routing inference outputs through structured inference and safety layers prior to physical actuation, the system preserves real-time adaptive capability while maintaining safety, regulatory compatibility, and operational reliability.

In some embodiments, the patient-state inference module 204 is configured to compare acquired procedural data, including data localized within an identified ROI, to one or more reference representations retrieved from the reference repository 212. The patient-state inference module 204 computes one or more similarity, quality, consistency, or deviation measures between the acquired procedural data and the reference representations using structural, statistical, feature-based, learned, probabilistic, or composite metrics. Based on the comparison, the patient-state inference module 204 determines whether one or more acceptance criteria are satisfied. Acceptance criteria can correspond to achievement of anatomical alignment, signal quality thresholds, geometric consistency, safety constraints, protocol-defined milestones, or other procedural objectives.

In some embodiments, the output of patient-state inference module 204 comprises a structured state representation including geometric, spatial, physiological, mechanical, contextual, and/or probabilistic parameters. Such state representation can be continuously updated during execution of the procedure as new sensor data is acquired. The structured state representation defines or constrains an operational adaptation space within which the adaptive control policy module 206 maps inferred patient state and evaluation results defining permissible and effective modifications to the operational adaptation space, which defines the permissible set of controllable device behaviors available under current anatomical, procedural, and safety conditions. These modifications are provided to device operation module 208 for iterative modification of controllable device parameters, motion, or behavior as described below. For example, when an acceptance criterion is satisfied, patient-state inference module 204 provides output to the adaptive control policy module 206 for mapping inferred patient state and evaluation results to an operational adaptation space defining permissible and effective modifications to device operation. If an acceptance criterion is not satisfied, patient-state inference module 204 provides output to the adaptive control policy module 206 that does not authorize progression of the medical procedure to a next state and/or instructs adaptive control policy module 206 to maintain current device operation.

As mentioned above, the adaptive control policy module 206 receives output from the patient-state inference module 204 and analyzes the output to map the inferred patient-state representations and associated evaluation results to an operational adaptation space defining permissible and effective modifications to operation of the robotic device 106, procedural devices 108, and/or other devices of system 100. As used herein, the term “operational adaptation space” refers to a structured, bounded, multidimensional parameter space representing the set of permissible device-state transformations available under current anatomical, procedural, and safety constraints. The adaptive control policy module 206 can modify operation of the robotic device 106 and/or procedural device 108 within the boundaries defined in the operational adaptation space.

The operational adaptation space can include adjustable device parameters, control policies, motion trajectories, interaction forces, sensing strategies, acquisition geometry, timing sequences, energy delivery profiles, or other operational characteristics governing device-patient interaction. The operational adaptation space may further include modification of physical interaction parameters, including contact force, pressure distribution, impedance interaction, compliance modulation, or other mechanical interaction characteristics between the device and the patient. The operational adaptation space may be constrained by inferred anatomical context, procedural protocol, safety constraints enforced by the safety monitoring module 210, device capability limitations, or human supervisory input. The operational adaptation space can be dynamically updated as inferred patient-specific state evolves, and may include multiple levels of abstraction, including low-level parameter tuning, mid-level motion refinement, and higher-level behavioral or strategic adjustments.

The adaptive control policy module 206 performs iterative modification of one or more controllable device parameters within the defined operational adaptation space in response to updated patient-state representations and evaluation outcomes. Iterative modification can include incremental adjustment, staged optimization, ordered parameter refinement, or policy-based reconfiguration of control objectives. In some embodiments, optimization or adjustment of one parameter can precede, inform, or constrain adjustment of another parameter, and such ordering can be predefined, dynamically determined, or influenced by supervisory input (e.g., from remote device 118 of human expert). In some embodiments, the adaptive control policy module 206 operates under different levels of autonomy, including human-guided, shared-control, semi-autonomous, or autonomous execution, subject to authorization and safety constraints imposed by other system components and/or modules (such as safety monitoring module 210).

Adaptive control policy module 206 also generates control instructions that are transmitted to device operation module 208. Device operation module 208 interfaces with robotic device 106 and/or procedural devices 108 to effect physical modification of device state in accordance with the generated control instructions. Such modification includes alteration of mechanical configuration, motion trajectory, force application, sensing behavior, acquisition parameters, or other machine-operational variables that govern device-patient interaction. In this manner, inferred patient state and evaluation outcomes are translated into concrete transformation of electromechanical or procedural device state during execution of the medical procedure.

For example, device operation module 208 is configured to generate and transmit control commands to one or more devices of system 100 that are either involved in, monitoring, or acquiring data relating to the medical procedure, including patient support structure 102, patient-wearable devices, robotic device 106 and corresponding sensing elements 106′, procedural devices 108, and environment sensors 110. In some embodiments, device operation module 208 translates high-level execution directives or candidate actions received from adaptive control policy module 206 into low-level control signals such as motion commands, force or torque limits, compliance parameters, or actuator setpoints. Also, device operation module 208 can operate in conjunction with real-time feedback from patient support structure 102, patient-wearable devices, robotic device 106 and corresponding sensing elements 106′, procedural devices 108, and environment sensors 110, and device operation module 208 can be configured to enforce control constraints defined by the adaptive control policy module 206 or safety monitoring module 210.

Safety monitoring module 210 is configured to independently monitor system operation and physical interaction conditions during execution of the medical procedure to detect safety-relevant events. Safety monitoring module 210 operates as a supervisory layer distinct from patient-state inference module 204, adaptive control policy module 206, and device operation module 208, and safety monitoring module 210 is configured to evaluate system behavior against predefined or dynamically determined safety constraints.

In some embodiments, safety monitoring module 210 functions as a safety and constraint enforcement layer that operates independently of patient-state inference module 204 and adaptive control policy module 206. The safety and constraint enforcement layer evaluates proposed adaptive modifications relative to predefined or dynamically determined operational bounds, including motion limits, force thresholds, energy delivery constraints, acquisition parameter ranges, and anatomical proximity limits. The enforcement layer may constrain or reshape the operational adaptation space prior to execution by device operation module 208.

In some embodiments, the safety monitoring module 210 receives inputs from data capture module 202, patient-state inference module 204, adaptive control policy 206, and/or device operation module 208 during execution of the procedure. Such inputs include sensor measurements, inferred patient-state representations, commanded control parameters, actuator states, force or torque values, motion trajectories, environmental conditions, and/or procedural phase indicators. Safety monitoring module 210 evaluates such inputs relative to safety envelopes, force thresholds, motion limits, anatomical proximity constraints, protocol-defined requirements, system integrity checks, or other risk-based criteria.

Upon detecting a safety condition—such as excessive force, unexpected motion, loss of sensor integrity, deviation from authorized operational limits, protocol violations, or system faults—safety monitoring module 210 is configured to generate one or more override or constraint signals. Such signals may restrict, bound, modify, interrupt, or terminate physical interaction between the robotic device 106 and the patient 104, irrespective of the current adaptive control state or level of autonomy. In some embodiments, the safety monitoring module 210 can temporarily suspend or revoke authorization for continued operation within the operational adaptation space defined by the adaptive control policy module 206.

In some embodiments, the safety monitoring module 210 transmits an alert notification to the remote computing device 118 operated by a human expert. The alert notification can be generated in response to detection of a safety condition, system fault, or protocol deviation and may include contextual information such as the nature of the detected condition, relevant sensor data, inferred patient-state parameters, system state information, and/or a severity indicator. Such notification enables the human expert to assess the situation and, where appropriate, provide supervisory input, modify acceptance criteria, adjust operational constraints, or issue override commands to the computing device 112. In some embodiments, human supervisory input influences region-of-interest definition, acceptance criteria thresholds, procedural phase progression, optimization ordering, or adaptation strategy, and can dynamically modify constraints applied within the operational adaptation space.

Also, in some embodiments, actions taken by the safety monitoring module 210 are architecturally prioritized over actions taken by the adaptive control policy module 206 and the device operation module 208. Generally, override or constraint signals generated by the safety monitoring module 210 may not be superseded by adaptive control logic, thereby ensuring that safety constraints remain enforceable independently of inference outcomes, reference evaluations, or optimization objectives.

For example, the system 100 supports controlled transition between autonomous adaptive control and manual or teleoperated control modes. Upon detection of non-satisfaction of acceptance criteria, repeated optimization failure, supervisory request, or safety-triggered event, authority for device control may be transferred from the adaptive control policy module 206 to a human operator. During manual control mode, the device operation module 208 executes operator-generated control inputs while continuing to acquire sensing data. The patient-state inference module 204 may continue to generate structured state representations during manual control. Upon completion of manual adjustments, control authority may be returned to the adaptive control policy module 206, and the adaptive pipeline resumes from the updated device state. This bidirectional authority transfer mechanism preserves procedural continuity while maintaining safety and human oversight.

FIG. 2 also illustrates a feedback loop interconnecting data capture module 202, patient-state inference module 204, adaptive control policy module 206, device operation module 208, and the devices 106, 108 of system 100. Through this feedback loop, each adaptive modification of device operation results in acquisition of updated sensing data, refreshed patient-state inference, and re-evaluation relative to stage-appropriate reference representations. In addition, the feedback loop enables continuous refinement of device behavior during execution of the medical procedure. As sensing data evolves in response to device movement, tissue interaction, environmental changes, or progression of procedural phase, the patient-state inference module 204 updates patient-state representations and recomputes evaluation metrics, and adaptive control policy module 206 correspondingly adjusts operational parameters within the defined adaptation space.

This closed-loop architecture allows device operational state to be iteratively transformed in real time without interruption or restart of the procedure, thereby enabling patient-specific adaptation to anatomical variability, interaction dynamics, and contextual changes occurring during execution. The feedback loop can operate continuously, periodically, or be event-triggered, and the feedback loop can be subject to operations performed by safety monitoring module 210 and/or optional human supervisory input as described herein.

In some embodiments, the system architecture generally comprises a sensing layer, a patient-state inference layer, a reference evaluation layer, an adaptive control layer defining an operational adaptation space, a safety and constraint enforcement layer, and an execution layer. The safety and constraint enforcement layer operates concurrently with adaptive control to ensure that all device-state transformations remain within clinically acceptable limits. Human supervisory interfaces may interact with one or more of these layers without disrupting closed-loop adaptive execution.

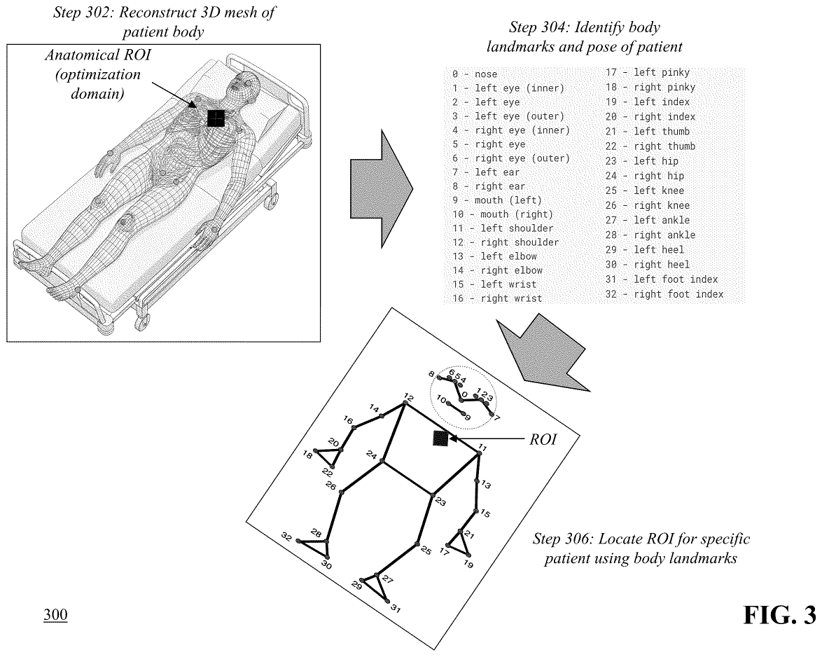

FIG. 3 is a flow diagram of a first stage 300 of patient-state inference in which external body configuration, surface landmarks, pose, or spatial orientation are estimated using sensing data to establish an initial spatial reference for adaptive control. This stage can be performed by patient-state inference module 204 based on data acquired through data capture module 202 from one or more environment sensors 110 (e.g., depth cameras, imaging sensors, or other sensing elements) positioned in or around the clinical environment. Acquired data can include, but is not limited to, depth maps, point clouds, stereo image pairs, structured light measurements, infrared imagery, or other surface-acquisition data capable of capturing three-dimensional geometry of the patient's external body surface. At step 302, the patient-state inference module 204 reconstructs a three-dimensional (3D) body mesh, skeletal model, or surface topology representation corresponding to the specific patient and current procedural posture using the acquired sensor data.

At step 304, based on the reconstructed 3D mesh representation, the patient-state inference module 204 identifies one or more anatomical body surface landmarks and estimates overall body pose. These landmarks can include, but are not limited to, head position, shoulder orientation, thoracic boundaries, rib cage contour, clavicular alignment, hip position, or other identifiable anatomical features. An exemplary list of body landmarks is provided in FIG. 3. In some embodiments, module 204 performs landmark detection using geometric fitting, feature extraction, model-based estimation, trained machine-learning models, statistical shape models, or combinations thereof. As can be appreciated, the resulting external body configuration representation establishes an initial spatial reference frame for subsequent adaptive control stages. In particular, the inferred pose and landmark information defines a coarse region-of-interest search space within which a target anatomical window or procedural access location is expected to reside.

At step 306, the patient-state inference module 204 locates an anatomical ROI for the specific patient based upon the previously inferred external body configuration, pose, and identified anatomical surface landmarks. In particular, the structured representation generated during the first-stage inference establishes a patient-specific spatial reference frame relative to the patient support structure 102 and the robotic device 106. Using this reference frame, the patient-state inference module 204 estimates the expected spatial location, orientation, and extent of a target anatomical ROI (also called a window or procedural access region).

In some embodiments, ROI localization is performed using geometric relationships between identified landmarks, statistical anatomical models, learned spatial mappings, or rule-based heuristics correlating external surface features with underlying anatomical structures. The resulting ROI definition can include one or more bounding volumes, surface patches, coordinate transforms, search corridors, or probabilistic spatial distributions representing the region within which a procedural objective is likely to be satisfied. The identified ROI then constrains subsequent adaptive control implemented by the adaptive control policy module 206 and/or the device operation module 208 by limiting permissible device motion, search trajectories, sensing focus, or parameter optimization within a patient-specific operational adaptation space.

In some embodiments, the adaptive control policy module 206 designates the anatomical region of interest (ROI) as a patient-specific localized optimization domain. The optimization domain refers to the ROI (or a localized subregion of the ROI) that is designated for optimization of device operating parameters. The optimization domain can be geometrically represented as a bounded region (e.g., rectangular, polygonal, elliptical, or freeform) aligned to anatomical landmarks or internal anatomical orientation inferred during earlier stages of patient-state inference. In one embodiment, the patient-specific localized optimization domain may be implemented as a Settings Selection Box (SSB). However, the optimization domain is not limited to a rectangular or explicitly bounded box and may comprise any spatially or functionally defined subregion of acquired data or inferred anatomical space.

The optimization domain functions as a constrained evaluation domain within which acquired data are compared to one or more reference representations. By restricting evaluation to the optimization domain, the system focuses optimization on diagnostically relevant structures rather than global image characteristics. In some embodiments, the optimization domain is predicted or estimated prior to physical interaction based on external configuration inference. In other embodiments, the optimization domain is not fully defined prior to scan initiation and is instead determined during execution of the procedure based on interaction-dependent sensing data, preliminary image acquisition, procedural context changes, or convergence behavior. In such embodiments, the optimization domain may emerge “just-in-time” as regions of elevated diagnostic relevance are detected and progressively refined through iterative evaluation.

In ultrasound embodiments, the localized optimization domain may correspond to a predicted or inferred acoustic window. An acoustic window refers to a patient-specific spatial region through which acoustic energy can propagate with sufficient quality to visualize a target anatomical structure. The acoustic window may be determined based on external body configuration, rib spacing, thoracic geometry, tissue composition, inferred internal anatomical orientation, or combinations thereof. For example, in an embodiment of a parasternal long-axis (PLAX) cardiac ultrasound procedure, the optimization domain may be aligned with the left ventricle and mitral valve region; in an apical four-chamber embodiment, the optimization domain may encompass ventricular cavities and atrioventricular valves.

In some embodiments, the optimization domain is dynamically updated as the inferred patient-specific state evolves. Alignment between external sensing data, inferred internal anatomical orientation, and probe pose may be refined iteratively, resulting in corresponding updates to optimization domain location, shape, or extent. The optimization domain may therefore move, rotate, scale, or otherwise adapt in response to updated inference results. In some embodiments, the optimization domain may initially encompass a broad search region and progressively narrow as confidence in anatomical alignment increases. In other embodiments, the optimization domain may be instantiated only upon detection of a candidate anatomical feature or quality threshold, rather than being predefined prior to evaluation.

The optimization domain is logically coupled to the adaptive control policy module 206. Image quality metrics, similarity scores, or other evaluation measures computed within the optimization domain are used to determine whether an acceptance criterion is satisfied and to guide optimization within the operational adaptation space. The optimization domain thereby serves as a structured interface between anatomical inference and device parameter adaptation. In some embodiments, the optimization domain may be presented to a human operator for confirmation, adjustment, or override. Operator-modified optimization domain boundaries may be incorporated into subsequent inference and adaptive control steps. By establishing a patient-specific coordinate system and landmark-based reference geometry, the patient-state inference module 204 reduces reliance on fixed geometric presets and enables downstream fine-grained optimization within a localized and patient-tailored search region.

In some embodiments, the patient-state inference module 204 is configured to estimate or initialize a patient-specific localized optimization domain prior to or during initial probe placement using two-dimensional (2D) and/or three-dimensional (3D) external imaging data acquired from environment sensors 110. In other embodiments, such estimation is deferred until interaction-dependent data is acquired, and the optimization domain is computed based on early-stage procedural sensing data rather than solely on external surface landmarks. The optimization domain may be predicted using geometric landmark relationships, learned spatial mappings between surface topology and internal anatomical orientation, regression models, or probabilistic spatial estimators. The predicted optimization domain may be represented as a bounding box, surface patch, volumetric region, or parametric search corridor within external anatomical space and mapped into device coordinate space. The optimization domain prediction constrains initial probe positioning and limits the operational adaptation space to a patient-specific sub-region likely to yield diagnostically relevant views.

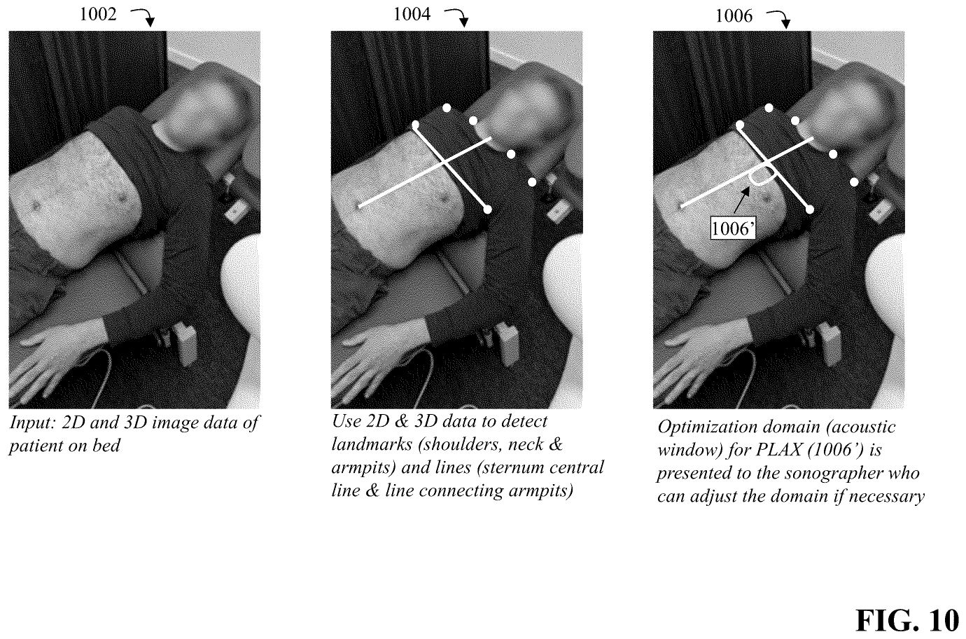

The patient-state inference module 204 may perform deterministic geometric estimation of an optimization domain using fused two-dimensional (2D) and three-dimensional (3D) imaging data acquired from environment sensors 110. For example, as illustrated in FIG. 10, the patient-state inference module 204 processes external imaging data received from environment sensors 110 (e.g., 2D and 3D image data of patient lying on bed 1002). As shown in annotated image 1004, the patient-state inference module 204 uses, e.g., computer vision algorithms to detect anatomical surface landmarks including, but not limited to, shoulders, neck base, axillary (armpit) locations, clavicular contours, and thoracic midline features. In some embodiments, landmark detection may be performed using feature extraction, depth-based segmentation, skeletal pose estimation models, statistical shape models, or combinations thereof.

Using the detected landmarks, the patient-state inference module 204 constructs one or more anatomical reference axes, as shown in annotated image 1006. In some embodiments, a longitudinal sternum midline axis is estimated based on detected clavicular alignment and thoracic symmetry, and a transverse axis is constructed by connecting left and right axillary landmarks. These axes define a patient-specific surface coordinate frame aligned with external anatomy.

Based on geometric relationships between the constructed axes and stored anatomical mappings corresponding to a candidate procedural target (e.g., a parasternal long-axis (PLAX) acoustic window), the patient-state inference module 204 may compute a predicted or provisional optimization domain region 1006′. The predicted region may be represented as a bounding box, parametric surface patch, angular sector, or volumetric search corridor mapped into device space. The predicted optimization domain constrains initial probe placement and limits the operational adaptation space prior to device-patient interaction. In some embodiments, the predicted region is provisional and subject to confirmation or refinement based on interaction-dependent sensing data acquired after probe contact. In some embodiments, the predicted optimization domain is presented visually to a human operator, who may confirm, translate, rotate, or resize the window prior to initiating probe contact. Adjustments made by the operator update the underlying coordinate transforms and corresponding constraints within the operational adaptation space.

In some embodiments, the optimization domain is not represented as a discrete geometric boundary but instead as a probabilistic weighting map or relevance field over sensing space. In such embodiments, evaluation metrics are spatially weighted according to dynamically inferred relevance scores, and the effective optimization domain corresponds to regions exceeding a threshold relevance criterion. This probabilistic or soft-domain formulation enables adaptive focus without requiring prior explicit geometric designation of a bounded region.

In some embodiments, the patient-state inference module 204 continuously or periodically updates the external body configuration inference during execution of the medical procedure. Changes in patient posture, involuntary movement, respiration, or support-surface adjustment can result in updated landmark estimates and corresponding adjustment of the spatial reference frame. Such updates are incorporated into the feedback loop described with respect to FIG. 2, enabling real-time refinement of ROI localization and adaptive device behavior.

In the illustrative example of FIG. 3, the patient-state inference module 204 locates a ROI comprising a parasternal long-axis (PLAX) acoustic window on the patient for the purpose of obtaining an echocardiogram ultrasound. However, other types of localization can be performed for other procedures without departing from the scope of the technology described herein. As shown in FIG. 3, the inferred external configuration includes estimates of patient orientation relative to, e.g., the patient support structure 102, angular displacement of the torso, elevation or rotation of anatomical segments, and spatial relationship between body landmarks (such as thoracic landmarks useful for localizing an echocardiogram ultrasound) and the robotic device 106 (and/or procedural devices 108). In some embodiments, the patient-state inference module 204 accounts for arbitrary patient positioning, including variations in body angle, limb placement, torso rotation, and anatomical proportion differences between patients, when locating the PLAX acoustic window for the specific patient.

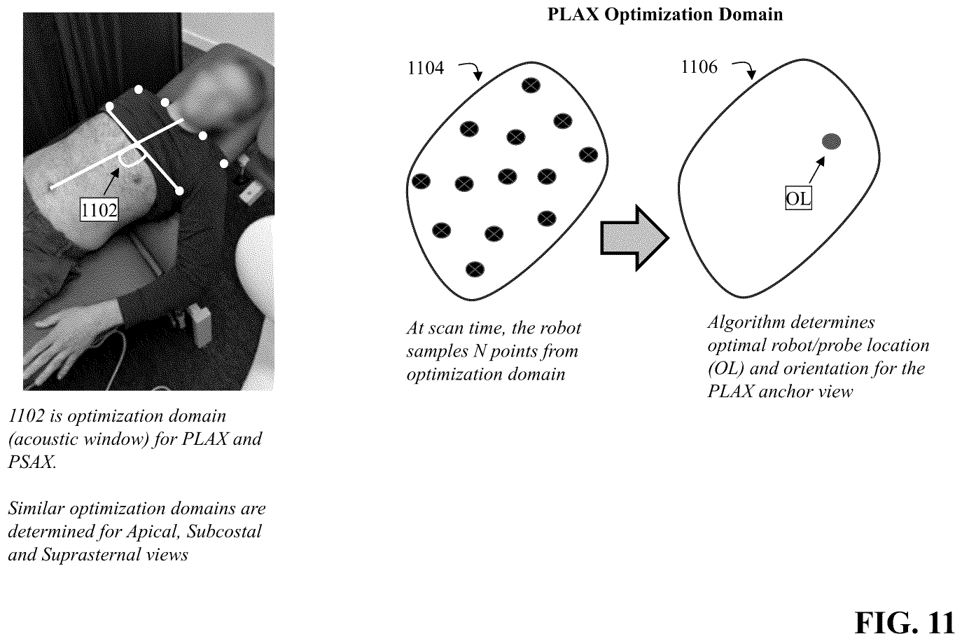

In some embodiments, following estimation of an optimization domain during first-stage external configuration inference, the adaptive control policy module 206 performs structured spatial sampling within the predicted optimization domain prior to determination of a final anchor probe pose. For example, as illustrated in FIG. 11, the predicted acoustic window 1102 may be represented as a bounded surface region defined in patient anatomical space and mapped into device space. The robotic device 106 positions the procedural device 108 at a plurality of discrete sampling points (1104) distributed across the window. In some embodiments, the sampling points are arranged according to a grid, quasi-random distribution, stratified sampling pattern, or adaptive exploration sequence within the window boundaries.

At each sampling location, the patient-state inference module 204 acquires ultrasound image data and associated interaction parameters, including probe pose, contact force, depth, and orientation. The acquired data is evaluated using one or more similarity or quality metrics relative to stored reference representations corresponding to the desired anchor view (e.g., PLAX anchor view). In some embodiments, the patient-state inference module 204 uses an algorithm (e.g., a regression-based estimation algorithm) to analyze the collected sampling data for determining an optimal probe location (reference character OL in FIG. 11) and orientation within the window (1106). The regression model may be deterministic and may estimate a pose vector that maximizes similarity to a reference representation or minimizes alignment error relative to canonical anatomical structures. Such regression can include linear regression, polynomial regression, multivariate regression, surface fitting, or other parameter-estimation techniques.

The estimated optimal probe pose defines an anchor view location within the optimization domain. The robotic device 106 then repositions the procedural device 108 to the estimated optimal pose and reacquires imaging data for confirmation. In some embodiments, the anchor view is presented to a human operator for approval. If the anchor view is not approved, the operator may manually adjust probe pose, and the updated pose may be incorporated into the structured patient-state representation and operational adaptation space. This structured sampling and regression-based optimization approach may be repeated for multiple optimization domains, including parasternal long-axis (PLAX) acoustic windows, parasternal short-axis (PSAX) acoustic windows, apical acoustic windows, subcostal acoustic windows, and suprasternal acoustic windows, thereby enabling protocol-compliant acquisition of standardized echocardiographic views.

In some embodiments, determination of an optimal probe pose within an optimization domain is preceded by a calibration procedure performed once per system configuration, per probe type, or per procedural protocol. During the calibration phase, the robotic device 106 systematically samples a plurality of probe positions and orientations within a defined optimization domain while acquiring ultrasound imaging data and associated interaction parameters, including contact force, applied pressure, and probe trajectory. For each sampled pose, one or more image quality metrics are computed relative to a canonical reference representation corresponding to a target anchor view (e.g., apical four-chamber or parasternal long-axis).

The calibration procedure characterizes the relationship between probe pose deviations and image quality degradation. In certain embodiments, this relationship is modeled using a regression algorithm that estimates a pose-quality response surface over the optimization domain. The regression model may be linear, polynomial, multivariate, or non-linear, and may incorporate position, orientation, and force components as independent variables.

The output of the calibration procedure comprises a fitted regression model that maps probe pose parameters to predicted image quality metrics. The model may compensate for variations in body type, anatomical orientation, optimization domain geometry, and typical heart position within the thoracic cavity.

At scan time, when N sampling points are acquired within the optimization domain for a particular patient, the calibration-derived regression model is applied to the sampled data to estimate an optimal probe position and orientation that maximizes predicted image quality for the target anchor view. The robotic device 106 then repositions the procedural device 108 accordingly. In some embodiments, calibration may be performed once during system setup, periodically, or upon detection of probe replacement or system configuration change. Calibration data and resulting model parameters may be stored in the reference repository 212 for subsequent retrieval during runtime optimization.

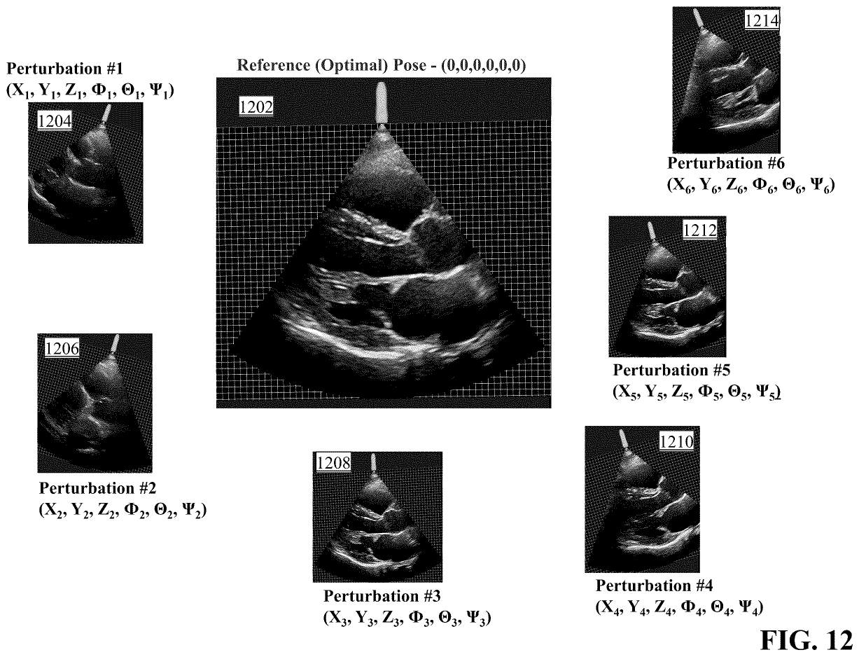

In some embodiments, probe pose optimization within an optimization domain is performed in a six-degree-of-freedom (6-DOF) parameter space comprising three translational components (X, Y, Z) and three rotational components (Φ, Θ, Ψ), corresponding to spatial position and orientation of the procedural device relative to the patient. As illustrated in FIG. 12, a reference optimal pose 1202 may be represented as a pose vector (0,0,0,0,0,0) in a local coordinate frame. Controlled perturbations of the probe pose (e.g., perturbations 1204, 1206, 1208, 1210, 1212, and 1214) may be generated by varying one or more translational or rotational components to produce perturbed poses (Xi, Yi, Zi, Φi, Θi, Ψi). For each perturbation, ultrasound image data and associated interaction parameters are acquired.

Image quality degradation or similarity variation resulting from pose perturbation may be quantified using one or more comparison metrics relative to a reference representation corresponding to a desired anchor view. The system thereby characterizes a local pose-quality response surface in the neighborhood of the reference pose. In some embodiments, a regression model, surface fitting algorithm, gradient estimation procedure, or other optimization method is applied to the perturbation data to estimate a pose update direction and magnitude that improves image quality. The optimization may be performed in full 6-DOF space or in constrained subspaces depending on anatomical context, procedural phase, or safety constraints.

In some embodiments, perturbations are applied sequentially, symmetrically, adaptively, or according to a predefined sampling strategy. Perturbation magnitudes may be bounded by safety limits, anatomical constraints, or device kinematic constraints defined within the operational adaptation space. This perturbation-based modeling enables fine-grained refinement of probe pose, compensating for patient-specific anatomical orientation, thoracic geometry, tissue compliance, and organ positioning variability.

Although FIG. 3 illustrates a specific embodiment involving 3D body reconstruction and landmark identification, it should be appreciated that an external configuration inference may be performed using any suitable sensing modality, computational approach, or representational form without departing from the scope of the invention. For example, the first-stage inference need not produce a full body mesh and can instead generate partial, probabilistic, feature-based, or reduced-order representations sufficient to establish a patient-specific spatial reference for adaptive control.

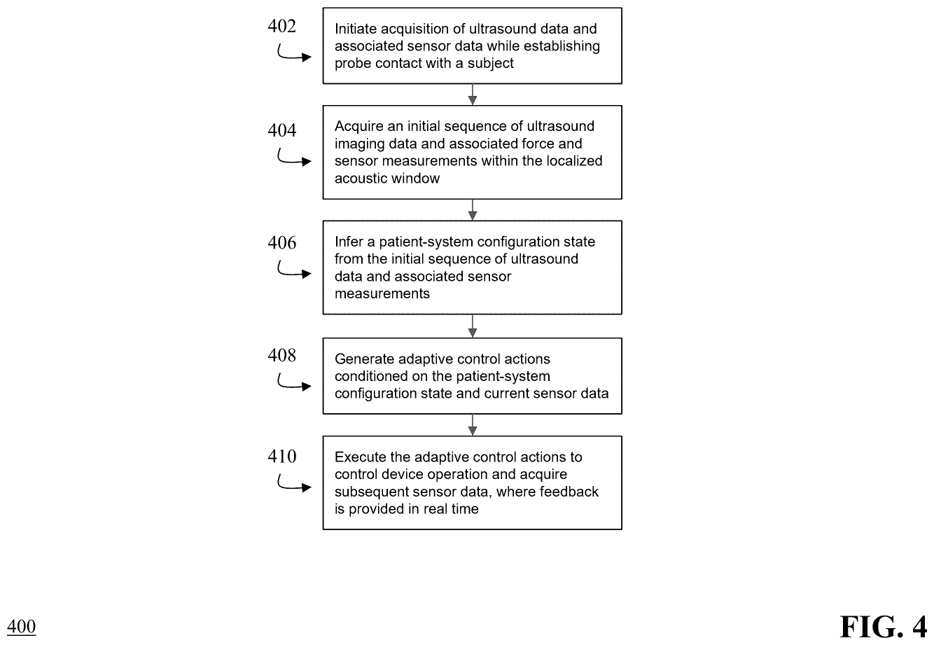

FIG. 4 is a flow diagram of a second stage 400 of patient-state inference in which device-patient interaction data is used to estimate internal anatomical orientation, spatial alignment between device and anatomy, or patient-specific interaction state relevant to a procedural objective. In some embodiments, steps of this second stage 400 are performed by one or more of the patient-state inference module 204, the adaptive control policy module 206, and the device operation module 208, following localization of an initial region of interest by module 204 as described above with respect to FIG. 3. Generally, in the second stage of patient-state inference, estimation of patient-system configuration is performed using data acquired during active physical interaction between the procedural device 108 and the patient 104. Unlike first-stage inference, which relies primarily on externally observed surface configuration, second-stage inference incorporates interaction-dependent signals reflecting mechanical coupling, acoustic transmission, impedance response, force-displacement behavior, or other device-tissue interaction phenomena.

At step 402, acquisition of procedural sensing data is initiated while establishing physical interaction between the procedural device 108 and the patient 104. In the illustrative echocardiogram ultrasound embodiment described herein, this may include initiating acquisition of ultrasound imaging data while establishing probe contact with the patient's skin. Concurrently, associated sensor data—such as force, torque, pose, orientation, contact pressure, impedance, or motion measurements—are acquired from the robotic device 106, device-mounted sensing elements 106′, and/or the procedural devices 108.

At step 404, an initial sequence of procedural data is acquired within the localized ROI established during first-stage inference. In some embodiments, the initial sequence includes a series of image frames, signal measurements, and associated interaction parameters collected while the procedural device 108 is positioned within a coarse anatomical window. As can be appreciated, the acquired data can reflect variations in tissue response, acoustic coupling, anatomical structures, device orientation, and applied force.

At step 406, the patient-state inference module 204 infers a patient-system configuration state from the acquired procedural data and associated sensor measurements. The inferred configuration state includes estimates of internal anatomical orientation relative to the device, alignment between the device sensing axis and a target anatomical structure, depth and orientation of tissue layers, probe-to-structure angle, quality of acoustic coupling, or other spatial or interaction parameters. In some embodiments, the patient-state inference module 204 further estimates contact conditions and tissue characteristics based on interaction data. Such estimation can include determination of contact force distribution, coupling quality, relative stiffness or compliance of contacted tissue, attenuation characteristics, impedance profiles, or deformation response. The interaction-dependent parameters can further include estimation of acoustic coupling quality between the probe and patient tissue, including detection of air gaps, gel distribution, or pressure-mediated impedance variation, which may directly influence subsequent adaptive control actions.

These interaction-dependent parameters can influence estimation of internal anatomical orientation and can be incorporated into the structured patient-system configuration state representation. For example, the inferred patient-system configuration state can parameterize or reshape the operational adaptation space, thereby restricting permissible device motion, orientation, or interaction forces to those consistent with the inferred internal anatomical alignment.

Also, in some embodiments, the inferred configuration state includes one or more coordinate transforms mapping device coordinates to patient anatomical coordinates. Such transforms represent angular offsets, translational displacement, depth alignment, or rotational misalignment between the sensing axis of procedural device 108 and a target anatomical structure. These transforms are continuously updated during iterative interaction and directly parameterize adaptive control actions within the operational adaptation space.

In some embodiments, inference is performed using trained machine-learning models (e.g., AI model 204a), geometric reconstruction techniques, signal feature extraction, statistical estimation methods, or combinations thereof. The inferred patient-system configuration state can be represented as a structured state vector including spatial transforms, angular offsets, depth estimates, contact metrics, signal-quality indicators, probabilistic confidence measures, or composite descriptors. This representation refines the spatial reference established in the first inference stage and provides a more precise estimate of the relative alignment between the procedural device 108 and the relevant internal anatomy. The patient-state inference module 204 sends the output to the adaptive control policy module 206.

At step 408, the adaptive control policy module 206 generates control actions conditioned on the inferred patient-system configuration state and current sensor data received from the patient-state inference module 204. The control actions include adjustments to pose of the robotic device 106 and/or the procedural device 108, angular orientation, applied force, compliance parameters, acquisition depth, scanning trajectory, or other controllable variables within the defined operational adaptation space. In some embodiments, control actions are selected to improve anatomical alignment, optimize signal quality, enhance image clarity, reduce tissue stress, or advance toward a defined procedural objective.

Also, in certain ultrasound embodiments, control actions include dynamic adjustment of probe contact force and applied pressure based on inferred patient-specific tissue compliance, acoustic coupling quality, rib shadowing effects, or image-quality metrics indicative of acoustic interference. For example, if bony interference or insufficient coupling is inferred, the adaptive control policy module 206 can increase applied force within safety bounds, modify probe angle to avoid rib occlusion, or adjust contact location within the localized ROI. The adaptive control policy module 206 sends the control actions to the device operation module 208.

At step 410, the adaptive control actions are executed by device operation module 208 to modify operation of the robotic device 106 and/or procedural device 108. In some embodiments, the adaptive control actions can also modify operation of one or more other elements of system 100, such as the patient support structure 102 and/or the environment sensors 110. Execution of the control actions by the device operation module 208 results in updated device-patient interaction conditions and acquisition of subsequent sensor data from the clinical environment by data capture module 202. The resulting sensor data is fed back to the patient-state inference module 204, thereby closing the feedback loop and enabling iterative refinement of internal anatomical alignment and interaction state in real time.

Through iterative execution of steps 404 to 410, the system 100 progressively refines alignment between the device and patient-specific internal anatomy within the localized ROI. In some embodiments, the second-stage inference transitions from coarse, externally referenced localization to fine-grained, interaction-informed estimation of internal anatomical configuration. The inferred device-patient interaction state can be evaluated relative to stage-specific reference representations stored in, e.g., the reference repository 212, to determine whether alignment, signal quality, or interaction metrics satisfy predefined or dynamically determined acceptance criteria. As can be appreciated, the process can continue until the acceptance criteria—such as anatomical alignment thresholds, signal-quality metrics, or protocol-defined conditions—are satisfied, at which point the system 100 maintains the optimized configuration or transitions to a subsequent procedural stage.

Although FIG. 4 illustrates an embodiment involving echocardiogram ultrasound acquisition and probe-based interaction, it should be understood that the second-stage inference framework is not limited to ultrasound imaging. Device-patient interaction data can include other types of diagnostic, therapeutic, and/or interventional procedures involving a variety of interactions, such as electrical impedance measurements, optical signals, force-displacement responses, physiological measurements, or other modality-specific interaction data suitable for inferring internal anatomical orientation or alignment in a patient-specific manner.

The system 100 then transitions to a third stage of patient-state inference where the adaptive control policy module 206 is configured to identify and/or refine one or more patient-specific regions of interest (ROI) based on outputs of patient-state inference module 204 and associated evaluation results. The ROI can correspond to a target anatomical structure, lesion, physiological feature, procedural access location, or other objective relevant to execution of the medical procedure.

In some embodiments, the ROI is defined according to one or more sources, including protocol-defined criteria, operator input, automated inference results, or reference representations retrieved from the reference repository 212. For example, the ROI can be defined coarsely during first-stage external configuration inference (see FIG. 3), further constrained during second-stage device-patient interaction inference (see FIG. 4), and subsequently refined during this third stage based on alignment metrics, signal quality measures, or acceptance criteria.

The adaptive control policy module 206 maps the ROI between multiple coordinate domains, including sensing space (e.g., image coordinates, depth maps, or signal acquisition space), device space (e.g., robotic joint coordinates, end-effector pose, or probe orientation), and inferred anatomical space (e.g., patient-specific coordinate frames established through multi-stage inference). The mappings can include one or more coordinate transforms, geometric correspondences, bounding volumes, surface patches, probabilistic spatial distributions, or parametric representations defining the spatial extent of the ROI.

In some embodiments, the adaptive control policy module 206 refines the ROI iteratively based on updated patient-state inference results and evaluation outcomes. As new sensing data is acquired and processed through the closed-loop architecture described herein, alignment between external sensing data, inferred internal anatomical orientation, and device pose is recalculated. Deviations between expected and observed anatomical features, signal characteristics, or interaction metrics can result in the adaptive control policy module 206 adjusting ROI boundaries, spatial transforms, or search corridors within the operational adaptation space. Also, refinement of the region of interest can progressively narrow or reshape the operational adaptation space to improve localization accuracy and optimize device operation within the patient-specific anatomical context.

The adaptive control policy module 206 therefore dynamically updates device motion constraints, sensing focus, acquisition parameters, or interaction objectives to maintain alignment with the evolving ROI. In some embodiments, refinement of the ROI reduces the permissible operational adaptation space to a localized and patient-specific region, thereby increasing localization accuracy and improving procedural efficiency. The ROI can evolve continuously during execution of the procedure as additional sensing data is acquired and incorporated into the patient-state representation. For example, minor changes in patient posture, respiration-induced motion, tissue deformation, or device repositioning may result in recalculation of spatial correspondences and updated ROI localization. Such updates are incorporated into the feedback loop connecting sensing, inference, evaluation, and adaptive control, enabling real-time refinement without interruption of the procedure.

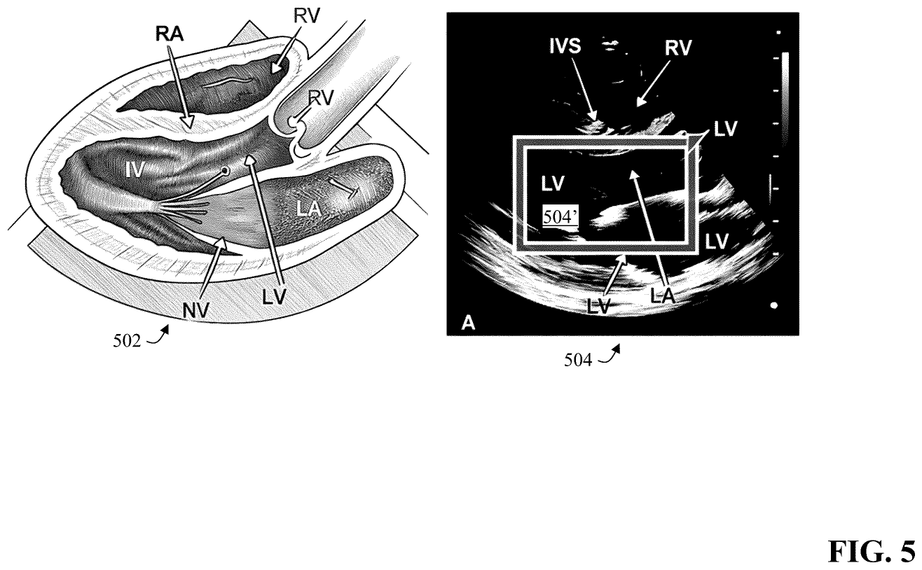

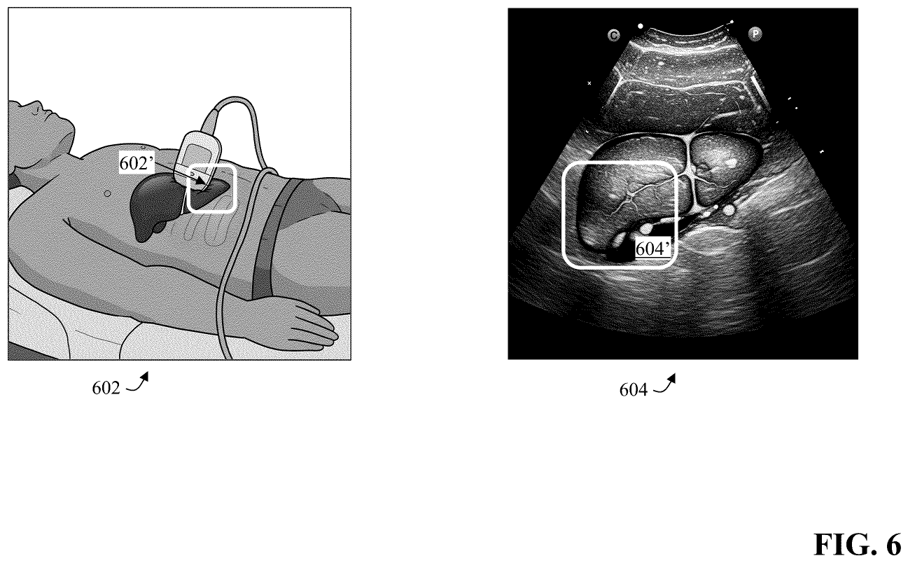

FIGS. 5 and 6 provide example embodiments of the third-stage ROI identification and refinement process performed by the adaptive control policy module 206. FIG. 5 illustrates the process in relation to optimization of ultrasound probe parameters for acquisition of a parasternal long-axis (PLAX) view in an echocardiogram ultrasound procedure. FIG. 5 depicts both an internal anatomical representation 502 and corresponding sensing data 504 captured by an ultrasound probe 108 (i.e., an ultrasound image), with a highlighted region (illustrated as bounding box 504′) representing the dynamically identified ROI.

As described above, the adaptive control policy module 206 utilizes outputs of the patient-state inference module 204 to establish a structured internal anatomical representation aligned with device coordinates. In FIG. 5, the highlighted region 504′ represents a patient-specific anatomical region selected for optimization based on alignment between: (i) externally inferred body configuration (Stage 1), (ii) device-patient interaction configuration state (Stage 2), and (iii) procedural objectives defined by protocol or reference representations retrieved from the reference repository 212.

In some embodiments, the ROI is defined in inferred anatomical space and mapped into both sensing space (e.g., ultrasound image coordinates) and device space (e.g., probe pose and orientation parameters). The bounded region 504′ illustrated in FIG. 5 corresponds to a spatially constrained volume or surface patch within which optimization of imaging parameters, probe angle, applied force, acquisition depth, and other controllable variables is performed. The mapping can involve coordinate transforms that align internal anatomical features (e.g., ventricular boundaries, valve structures) with the sensing axis and actuation parameters of the procedural device 108.

The adaptive control policy module 206 iteratively refines the ROI as updated sensing data is acquired. For example, deviations between expected anatomical structures (based on reference representations) and observed imaging data can result in adjustment of the ROI boundaries, rotation of the bounding region, translation within anatomical space, or modification of the associated operational adaptation space. In this manner, alignment between external sensing data, inferred internal anatomy, and device pose is dynamically improved.

The ROI 504′ illustrated in FIG. 5 therefore represents not merely a static geometric box, but a dynamically evolving, patient-specific region defined across multiple coordinate domains and updated through the closed-loop architecture described herein. As device operation module 208 modifies probe position or orientation and new data is captured by computing device 112, the patient-state inference module 204 updates the internal anatomical representation, evaluation relative to reference data is recomputed, and the adaptive control policy module 206 correspondingly refines the ROI.

FIG. 6 illustrates an additional example embodiment of third-stage region-of-interest (ROI) identification and refinement in the context of ultrasound imaging of the liver. In this embodiment, the ROI corresponds to a patient-specific anatomical region selected to optimize ultrasound probe parameters for acquisition of diagnostically relevant liver images.

As shown in the left portion 602 of FIG. 6, an external representation of patient 104 is illustrated, including a depiction of probe placement on the abdominal surface. A highlighted region 602′ (illustrated as a bounding box) represents the externally localized area corresponding to the inferred internal anatomical target. This external localization can be derived from first-stage external body configuration inference, including estimation of torso orientation, rib boundaries, abdominal surface topology, and spatial alignment relative to the patient support structure 102 and/or the robotic device 106.

The right portion 604 of FIG. 6 illustrates a corresponding ultrasound image in sensing space, with a highlighted region 604′ representing the patient-specific ROI aligned with internal liver anatomy. As described above, the patient-state inference module 204 and the adaptive control policy module 206 cooperate to map the ROI between external anatomical space, inferred internal anatomical space, and sensing space (e.g., ultrasound image coordinates). The mapping can include one or more coordinate transforms relating probe pose and orientation in device space to anatomical structures visualized in imaging space.

In some embodiments, the ROI 604′ corresponds to a specific hepatic segment, vascular structure, lesion, or tissue region of diagnostic interest. The ROI can be initially defined based on protocol, clinician input, or automated inference and subsequently refined based on alignment between observed imaging features and reference representations retrieved from the reference repository 212. For example, alignment metrics comparing expected liver contour, echogenic patterns, or vessel orientation with acquired ultrasound data may inform adjustment of ROI boundaries or device pose.

As described above, the adaptive control policy module 206 maps the refined ROI to an operational adaptation space defining controllable aspects of device operation. Such controllable aspects include probe position, orientation, tilt angle, applied force, scanning trajectory, acoustic frequency selection, focal depth, gain parameters, or other acquisition settings. As the ROI is refined iteratively, the permissible operational adaptation space is constrained to improve localization accuracy and optimize imaging quality within the patient-specific region.

As with the embodiment of FIG. 5, the ROI 604′ illustrated in FIG. 6 is not static. As additional sensing data is acquired and processed through the closed-loop architecture described herein, alignment between external localization, inferred internal anatomy, and device pose may be recalculated. The ROI can be translated, rotated, resized, or otherwise modified in response to updated inference results, tissue motion (e.g., respiration), or evaluation outcomes. In this manner, device behavior is adaptively optimized for the specific patient and procedural objective.

Although FIG. 5 illustrates an embodiment involving ultrasound imaging and PLAX acoustic window localization and FIG. 6 illustrates an embodiment involving ultrasound imaging of the liver, it should be appreciated that the third-stage ROI identification and refinement process is not limited to ultrasound procedures. The ROI can correspond to any anatomical structure, lesion, physiological feature, or procedural objective for which patient-specific alignment and parameter optimization are desirable.

Having described identification and refinement of a patient-specific region of interest with respect to FIGS. 5 and 6, FIG. 7 illustrates the reference-guided evaluation and acceptance-based optimization process through which adaptive control policy module 206 determines whether current device operation satisfies procedural objectives or requires further modification.

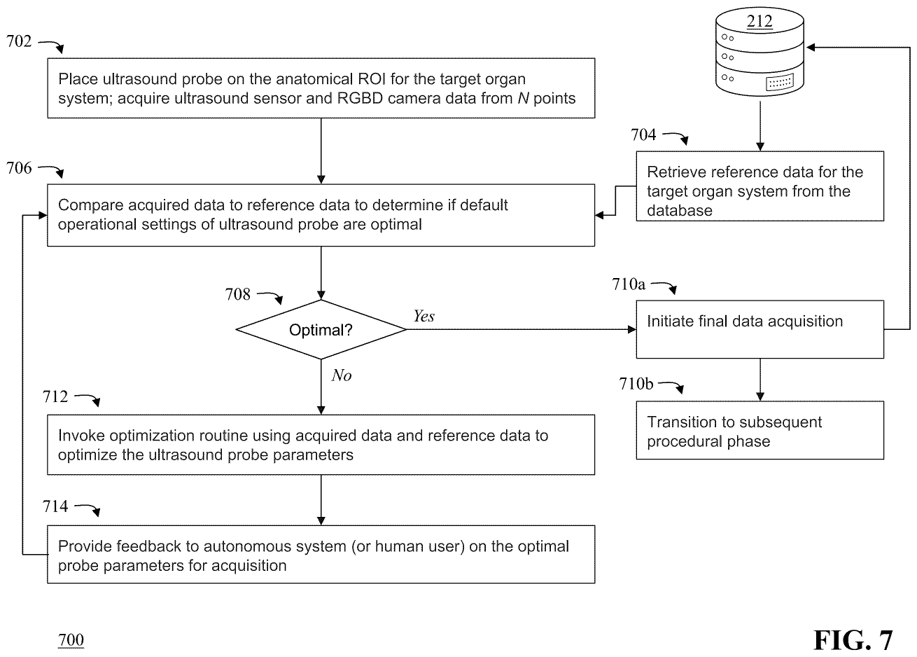

FIG. 7 is a flow diagram of a method 700 for comparing acquired procedural data within a region of interest to reference representations using similarity or quality evaluation metrics to determine whether an acceptance criterion is satisfied. In some embodiments, the steps of method 700 are performed by the patient-state inference module 204, including retrieval of stage-appropriate reference representations from the reference repository 212 and evaluation of acquired data from data capture module 202 relative to one or more acceptance criteria.

At step 702, the procedural device 108, such as an ultrasound probe, is positioned by the robotic device 106 and the computing device 112 within a patient-specific region of interest identified as described above. The procedural device 108 acquires imaging data, device pose information, force measurements, RGB-D sensing data, and other outputs from one or more acquisition points within the ROI. In some embodiments, the acquired data reflects current probe alignment, interaction conditions, anatomical visualization quality, or other measurable characteristics.

At step 704, the patient-state inference module 204 retrieves reference data associated with a target organ system, anatomical configuration, procedural phase, or optimization objective from the reference repository 212. As mentioned above, the reference data includes one or more reference representations such as anatomical templates, expected structural alignments, quality exemplars, signal characteristics, learned statistical models, or predefined protocol-defined targets. In some embodiments, retrieval of reference data is conditioned on inferred patient-specific state, procedural stage, identified region of interest, or operator input.

At step 706, the patient-state inference module 204 compares the acquired data to reference representations using one or more similarity, quality, or consistency measures. Such measures include structural similarity metrics, geometric alignment metrics, signal-to-noise ratios, learned evaluation models, probabilistic likelihood scores, or composite performance indicators.

At decision point 708, the patient-state inference module 204 determines whether an acceptance criterion has been satisfied based upon the comparison.

If the acquired data satisfies the acceptance criterion, the patient-state inference module 204 can initiate final data acquisition (step 710a), transition to a subsequent procedural phase (step 710b), or maintain the current robotic device 106/procedural device 108 configuration. In some embodiments, satisfaction of the acceptance criterion authorizes progression within the operational adaptation space and can trigger execution of a finalized acquisition sequence.

If the acceptance criterion is not satisfied, the patient-state inference module 204 instructs the adaptive control policy module 206 to invoke an optimization routine within the operational adaptation space. At step 712, using the acquired data and corresponding reference representations, the adaptive control policy module 206 (in conjunction with the device operation module 208) adjusts probe position, orientation, applied force, acquisition depth, focal parameters, gain settings, scanning trajectory, or other controllable aspects of device behavior. It should be appreciated that this optimization process can be automated, semi-autonomous, or guided by human supervisory input.

At step 714, the adaptive control policy module 206 generates feedback relating to one or more optimal probe parameters determined through the optimization routine. The feedback can include recommended adjustments to probe pose, angular orientation, contact force, depth setting, focal zone placement, gain profile, acoustic frequency selection, or other acquisition parameters. In some embodiments, the feedback may be presented to a human operator via a graphical user interface, visual overlay, haptic cue, auditory signal, or textual instruction indicating directional correction (e.g., rotate clockwise, increase force, translate laterally). In other embodiments, the feedback is transmitted directly to device operation module 208 to implement autonomous or semi-autonomous parameter updates within the constrained operational adaptation space, resulting in modified operation of the robotic device 106 and/or the procedural device 108.

Following adjustment of device parameters and delivery of feedback, the patient-state inference module 204 receives additional sensing data from the data capture module 202 and the evaluation process is repeated, thereby forming an iterative loop. Through repeated retrieval, comparison, and adaptation, the modules of the computing device 112 converge toward a patient-specific configuration that satisfies defined acceptance criteria.

In some embodiments, prior to acquisition of a full set of protocol-defined imaging views associated with a given optimization domain, the system identifies and evaluates a canonical anchor view. The anchor view corresponds to a predefined anatomical alignment or imaging orientation that serves as a reference configuration for subsequent view acquisition. The patient-state inference module 204 determines whether the anchor view satisfies one or more anchor-specific acceptance criteria based on comparison to stored reference representations. The anchor view may be evaluated using geometric alignment metrics, structural similarity metrics, or learned evaluation models. If the anchor view satisfies the acceptance criteria, the adaptive control policy module 206 authorizes acquisition of additional protocol-defined views within the same optimization domain. If the anchor view does not satisfy acceptance criteria, iterative optimization within the operational adaptation space is invoked until the anchor configuration is achieved or supervisory override is engaged. In some embodiments, the anchor view approval may require human confirmation prior to progression to multi-view acquisition.

Although FIG. 7 illustrates an embodiment involving ultrasound imaging optimization, the reference retrieval and evaluation process is applicable to any diagnostic or interventional modality in which acquired data can be evaluated relative to stored reference representations to guide adaptive device control.

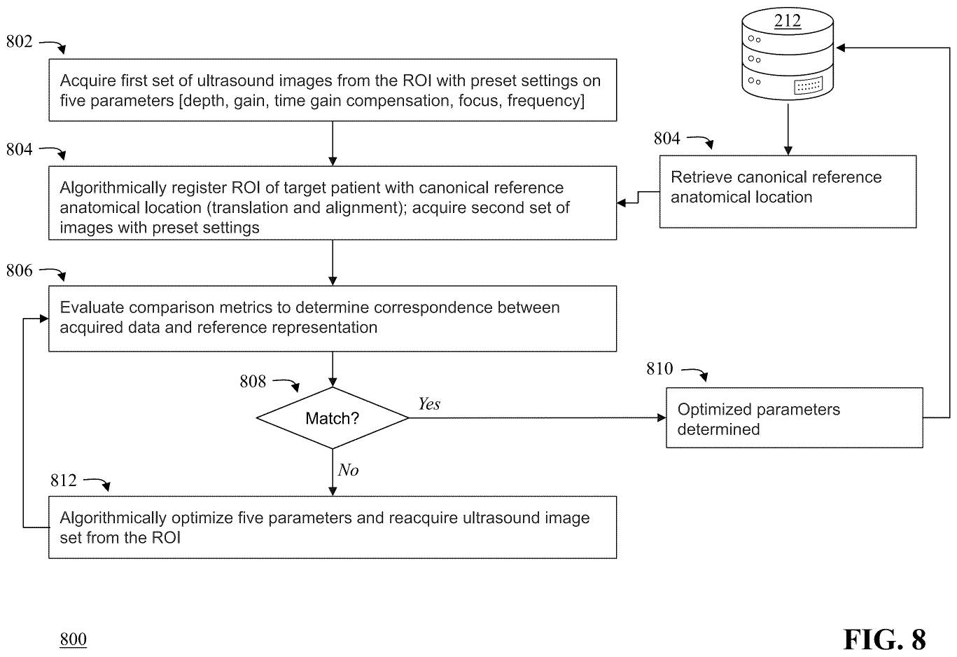

FIG. 8 is a flow diagram of a method 800 for determining satisfaction of an acceptance criterion based on a comparison between acquired data and reference representations. FIG. 8 provides a more detailed example of the evaluation and optimization logic (steps 706 and 708) described above with respect to FIG. 7.

At step 802, the patient-state inference module 204 acquires a first set of ultrasound images from the ROI using preset values for a plurality of probe parameters. In the illustrated embodiment, five parameters are evaluated, including imaging depth, overall gain, time gain compensation (TGC), focal position, and acoustic frequency. It should be appreciated that any number or type of controllable parameters may be used without departing from the scope of the invention.

At step 804, the patient-state inference module 204 retrieves a canonical reference anatomical location associated with the target organ system or procedural objective from the reference repository 212. In some embodiments, the canonical reference includes a geometric template, anatomical alignment model, expected structural configuration, learned statistical representation, or quality exemplar corresponding to a desired imaging state. In some embodiments, retrieval of the canonical reference is conditioned on the inferred patient-specific state, procedural phase, or identified ROI.

At step 806, the patient-state inference module 204 algorithmically registers the acquired ROI data to the canonical reference anatomical location. For example, registration can include translation, rotation, scaling, or other alignment operations to establish correspondence between the acquired ultrasound data and the reference representation.

At step 808, following registration, the patient-state inference module 204 evaluates one or more comparison metrics to determine correspondence between the acquired data and the reference representation. The comparison metrics can include structural similarity measures, anatomical alignment scores, signal-quality indicators, learned evaluation model outputs, or composite metrics.

In some embodiments, the comparison metrics evaluated at step 808 may be implemented using machine learning (ML)-based techniques, non-machine-learning-based techniques, or combinations thereof. ML-based metrics can include outputs of trained neural networks, convolutional models, transformer-based architectures, learned feature embeddings, similarity networks, probabilistic classifiers, or other data-driven models configured to estimate correspondence, alignment quality, anatomical consistency, or image adequacy relative to reference representations. The models can be trained using supervised, semi-supervised, unsupervised, or reinforcement learning approaches and output scalar quality scores, confidence measures, probability estimates, or multi-dimensional similarity descriptors. In some embodiments, models are periodically retrained or updated using newly acquired procedure data stored in the reference repository 212, subject to validation and regulatory constraints.

In other embodiments, the comparison metrics are non-ML-based and include deterministic or statistical similarity measures computed directly from image or signal data. Examples include, but are not limited to, structural similarity metrics (e.g., structural similarity index (SSIM)), normalized cross-correlation, mutual information, entropy-based measures, gradient-based similarity measures, histogram comparisons, edge alignment metrics, geometric alignment error, signal-to-noise ratio calculations, contrast measures, or other mathematically defined similarity functions. Mutual information-based metrics can quantify statistical dependence between acquired and reference representations, while structural similarity index metrics may evaluate luminance, contrast, and structural correspondence between image regions. As can be appreciated, the metrics can be computed on full images, localized regions of interest, extracted feature maps, or transformed representations. In some embodiments, hybrid approaches can be employed in which the patient-state inference module 204 combines deterministic similarity measures with learned models to improve robustness, generalization, or convergence performance. The specific comparison metric or combination of metrics used to evaluate satisfaction of the acceptance criterion may be selected based on modality, anatomical target, computational constraints, or procedural objectives.

At decision point 810, the patient-state inference module 204 determines whether the computed comparison metrics satisfy one or more predefined or dynamically determined acceptance criteria. If the acceptance criterion is satisfied, the patient-state inference module 204 confirms that optimized probe parameters have been determined (step 812). In some embodiments, step 810 may include confirmation that the current parameter configuration falls within acceptable tolerance bounds, storage of the optimized parameter set in, e.g., database 116′, and authorization of final data acquisition.

In some embodiments, estimation of an optimal probe pose within a patient-specific optimization domain is performed using deterministic regression techniques based on an initial set of sampled acquisition points. For example, the patient-state inference module 204 may acquire procedural data at multiple discrete probe positions within the predicted optimization domain and compute a regression model correlating pose parameters with one or more image-quality or anatomical alignment metrics. The regression model may include linear regression, polynomial regression, spline fitting, or other parametric curve-fitting methods configured to estimate a pose that maximizes correspondence relative to a reference representation. The estimated optimal pose is mapped into the operational adaptation space and used to generate control signals that reposition the procedural device accordingly. This regression-based estimation may be performed prior to fine-grained parameter optimization to accelerate convergence within the bounded adaptation space.

If the acceptance criterion is not satisfied, the patient-state inference module 204 proceeds to step 814, where the module 204 instructs the adaptive control policy module 206 to algorithmically optimize one or more of the probe parameters within the operational adaptation space. In some embodiments, optimization includes adjustment of depth, gain, TGC profile, focal position, frequency, probe pose, interaction force, or other controllable variables. Following optimization, a new set of ultrasound images is reacquired from the ROI, and steps 808 and 810 are repeated.

Through this iterative loop of acquisition, registration, evaluation, and parameter optimization, the system 100 converges toward a patient-specific configuration that satisfies the acceptance criterion. In some embodiments, the optimization process can be fully automated, semi-autonomous, or accompanied by feedback to a human operator as described above with respect to step 714 in FIG. 7. Although FIG. 8 illustrates an example of optimization of ultrasound probe parameters, the acceptance-based evaluation and iterative adaptation framework described herein is applicable to any diagnostic or interventional modality in which controllable device parameters are refined based on comparison of acquired data to canonical or reference representations.

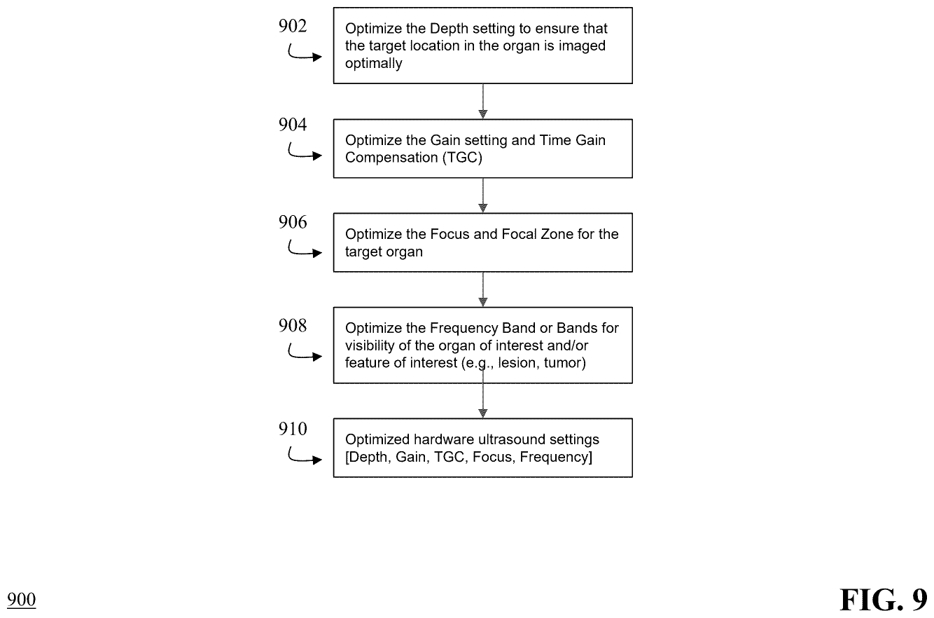

To further illustrate the parameter-optimization process (step 812 of FIG. 8) performed by the adaptive control policy module 206 when the acceptance criterion is not satisfied, FIG. 9 is a flow diagram of a method 900 of staged or ordered adaptive optimization of ultrasound device parameters within the operational adaptation space.

At step 902, the adaptive control policy module 206 optimizes an imaging depth parameter to ensure that a target anatomical location within the organ of interest is captured within the imaging field and positioned appropriately relative to the sensing window. In some embodiments, depth optimization involves adjusting imaging range to ensure inclusion of relevant anatomical structures while minimizing unnecessary acquisition of non-target regions.

At step 904, the adaptive control policy module 206 optimizes gain and time gain compensation (TGC) parameters to improve image contrast, dynamic range utilization, and signal clarity across varying tissue depths. In some embodiments, optimization of gain and TGC can be based on quantitative image-quality metrics, statistical intensity distributions, or learned evaluation outputs derived from the comparison metrics described above with respect to FIG. 8.

At step 906, the adaptive control policy module 206 optimizes focal position and focal zone parameters to improve resolution at the target anatomical region. In some embodiments, adjustment of focal depth is guided by inferred internal anatomical orientation, estimated tissue depth, or reference-aligned anatomical models retrieved from the reference repository 212.

At step 908, the adaptive control policy module 206 optimizes acoustic frequency band selection to enhance visualization of the organ or feature of interest (e.g., lesion, tumor, vascular structure). In some embodiments, frequency optimization balances penetration depth and spatial resolution based on patient-specific tissue characteristics inferred during earlier stages of patient-state inference. Also, in certain embodiments, staged optimization follows a dependency-aware ordering in which depth is optimized prior to gain adjustment, gain is optimized prior to focal refinement, and focal parameters are optimized prior to frequency selection, thereby reducing parameter interaction effects and improving convergence efficiency within the operational adaptation space.

Upon completion of the staged optimization sequence, the adaptive control policy module 206 has determined a set of optimized hardware ultrasound settings (step 910). In some embodiments, these optimized settings may comprise a parameter vector including depth, gain, TGC, focal configuration, and frequency values that collectively satisfy or improve correspondence relative to the canonical reference representation.

Although FIG. 9 illustrates a sequential optimization process in which parameters are refined in a predefined order, it should be appreciated that optimization may alternatively occur in parallel, iteratively, hierarchically, or adaptively based on evaluation feedback. In some embodiments, optimization of one parameter can influence constraints or search bounds for subsequent parameters within the operational adaptation space. The illustrated sequence therefore represents one example implementation of the broader parameter-optimization routine invoked at step 812.

Following determination of the optimized parameter set, the computing device 112 can reacquire imaging data and re-evaluate correspondence as described above with respect to FIG. 8, thereby maintaining the closed-loop architecture in which acquisition, evaluation, and adaptation are iteratively performed until satisfaction of the acceptance criterion.

As described herein, embodiments of the methods and systems are configured to utilize AI models 204a as part of the overall control framework. In particular, model inference can be performed within the patient-state inference module 204 of computing device 112 using live data streams acquired from robotic device 106, procedural devices 108, joint-level sensing elements 106′, and environment sensors 110. Model outputs are therefore directly grounded in physical measurements corresponding to device-patient interaction.

As illustrated in FIG. 2, AI model(s) 204a operate as subcomponents of patient-state inference module 204 and generate structured state representations that parameterize the operational adaptation space mapped by adaptive control policy module 206. The structured state representations produced by the trained models include spatial transforms, anatomical alignment parameters, tissue compliance estimates, signal-quality metrics, confidence measures, and/or candidate adaptation vectors. These outputs are not terminal results; rather, they are intermediate representations that are consumed by, e.g., the adaptive control policy module 206 and subsequently translated by the device operation module 208 into concrete control signals that modify actuator states, probe pose, interaction force, acquisition settings, or energy delivery parameters.

In the first-stage inference embodiment illustrated in FIG. 3, trained AI model(s) 204a can be configured to perform surface landmark detection, pose estimation, skeletal reconstruction, or probabilistic anatomical mapping using multi-modal environment sensor data. The output of the model defines a patient-specific spatial reference frame that directly constrains motion of the robotic device 106 by limiting permissible search regions for region-of-interest (ROI) localization. Thus, model inference results in bounded modification of robot motion planning within device space.

In the second-stage device-patient interaction inference illustrated in FIG. 4, trained AI model(s) 204a can estimate internal anatomical orientation, acoustic coupling quality, force-displacement response, tissue stiffness gradients, or alignment between sensing axes and target anatomy. These estimates parameterize coordinate transforms and constrain allowable probe orientation, applied force, scanning trajectory, or impedance interaction within the operational adaptation space defined by adaptive control policy module 206. Execution of control actions by device operation module 208 therefore produces measurable physical transformation of device state based on the model-derived inference outputs.

In the ROI refinement embodiments of FIGS. 5-6, trained AI model(s) 204a can generate feature embeddings or segmentation maps identifying anatomical substructures within acquired imaging or sensing data. These model outputs are mapped across sensing space, anatomical space, and device space to dynamically reshape the ROI boundaries. As the ROI is refined, permissible motion envelopes and parameter search bounds within the operational adaptation space are correspondingly narrowed. This dynamic reshaping directly modifies actuation constraints applied to the robotic device 106.

In the reference-based evaluation process illustrated in FIGS. 7-8, trained AI model(s) 204a can implement learned similarity networks, quality estimators, probabilistic classifiers, or composite evaluation models configured to compute correspondence between acquired data and reference representations retrieved from the reference repository 212. The scalar or vector outputs of such models can be evaluated against acceptance criteria at decision point 810. Satisfaction or non-satisfaction of acceptance criteria directly determines whether the adaptive control policy module 206 authorizes progression to finalized acquisition (step 812) or invokes parameter optimization (step 814), resulting in further device-state modification.

In the staged optimization embodiment illustrated in FIG. 9, trained model outputs may inform dependency-aware ordering of parameter refinement. For example, a model-estimated depth error may constrain subsequent gain adjustment bounds; a model-estimated tissue attenuation profile may inform focal zone selection; and a model-estimated compliance parameter may bound applied force. The staged sequence of depth, gain, focal configuration, and frequency optimization therefore reflects structured interaction between learned inference outputs and hardware-level parameter control.

In each of the foregoing embodiments, AI model inference is therefore concretely tied to (i) physical sensor acquisition, (ii) structured state representation generation, (iii) mapping to a bounded operational adaptation space, (iv) safety-constrained authorization, and (v) hardware-level actuation resulting in measurable physical transformation of device configuration, interaction force, sensing configuration, or energy delivery. The machine-learning components are not used to generate abstract recommendations in isolation, but instead function as integrated computational elements within a closed-loop electromechanical control architecture that continuously modifies physical device state during interaction with a human body.

By explicitly coupling trained model inference to physical sensing inputs and bounded electromechanical actuation outputs across the illustrated embodiments, the disclosed systems and methods provide a concrete technological improvement in medical device control systems. The integration of trained inference models within the structured sensing-inference-evaluation-adaptation-execution feedback loop enables patient-specific transformation of hardware operational state in real time, rather than mere post hoc data analysis or abstract decision support.

Also, while ultrasound imaging is described herein as an illustrative embodiment, the adaptive control framework applies broadly to non-invasive and minimally invasive diagnostic and interventional procedures involving controlled physical interaction between a medical device and a human body. The structured patient-state inference, region-of-interest localization, reference-based evaluation, and iterative adaptive control architecture described above may be instantiated across a wide range of modalities and device types without modification to the core closed-loop control logic.

Energy Delivery Systems—in some embodiments, the adaptive control framework is applied to therapeutic energy delivery systems, including but not limited to ultrasound ablation, radiofrequency ablation, laser therapy, electrical stimulation, cryotherapy, or focused energy delivery procedures. The patient-state inference techniques described herein can estimate tissue impedance, perfusion characteristics, thermal response, anatomical proximity to sensitive structures, or spatial alignment relative to a target lesion. Reference representations can include target energy distribution profiles or safety envelopes. Adaptive control can modify energy intensity, pulse duration, beam focus, delivery trajectory, contact force, or dwell time within the operational adaptation space to achieve a therapeutic objective while maintaining safety constraints.

Mechanical Assistive and Rehabilitation Systems—in some embodiments, the adaptive control framework can be applied to robotic rehabilitation devices, assistive exoskeletons, physical therapy systems, or mobility-support devices. The patient-state inference techniques described herein can estimate joint orientation, muscle activation, force output, fatigue state, or biomechanical alignment. Reference representations can correspond to desired motion trajectories, range-of-motion targets, gait patterns, or therapeutic exercise profiles. Adaptive control can iteratively adjust applied torque, resistance levels, motion amplitude, support force, or actuation timing based on patient-specific biomechanical response.

Image-Guided Localization and Interventional Navigation—in some embodiments, the adaptive control framework can be applied to image-guided needle placement, catheter navigation, biopsy targeting, or minimally invasive localization procedures. Multi-stage inference can estimate external surface landmarks, internal anatomical orientation, and target structure localization. Reference-based evaluation can assess alignment between device trajectory and target anatomy. Adaptive control can iteratively modify insertion angle, depth, trajectory curvature, or applied force within a constrained operational adaptation space until alignment criteria are satisfied.

Physiological Monitoring and Tactile Sensing Procedures—in some embodiments, the adaptive control framework can be applied to physiological monitoring procedures requiring adaptive sensor placement or contact optimization, including electrocardiography (ECG), electromyography (EMG), blood oxygen monitoring, impedance measurement, or tactile palpation systems. Patient-state inference may estimate signal quality, contact integrity, tissue compliance, motion artifacts, or electrode positioning accuracy. Adaptive control may adjust sensor placement, applied pressure, electrode configuration, sampling parameters, or filtering characteristics to achieve predefined quality or stability criteria.

Palpation and Tactile Sensing—in some embodiments, the adaptive control framework is instantiated as a system for autonomous robotic palpation and tactile sensing for detection of anatomical abnormalities, stiffness gradients, or sub-surface masses. A robotic device equipped with a physical interaction interface performs controlled probing of a target anatomical region. In this embodiment, sensing elements can include one or more six-degree-of-freedom (6-DOF) force/torque sensors, displacement sensors, compliance sensors, and optical or depth sensors configured to map surface topology and measure tissue deformation in response to applied force. Sensor data can include force-displacement curves, torque vectors, deformation gradients, and spatial surface reconstructions. Patient-state inference processes the relationship between applied force and measured deformation to infer patient-specific tissue properties, including stiffness gradients, localized compliance anomalies, viscoelastic response, or the presence of deep-seated nodules. In some embodiments, probabilistic models or machine learning models estimate mass boundaries or abnormal tissue regions based on spatially varying resistance profiles. Adaptive control maps the inferred tissue state to an operational adaptation space defining permissible probing depth, contact pressure limits, exploration trajectories, and motion velocities. Control actions may include modifying probing depth, adjusting contact force thresholds, altering exploration path density, or refining local search trajectories. In some embodiments, the system performs perturbation-based exploration to refine boundaries of a detected mass or abnormality. Controlled variations in contact angle, applied force vector, or probing direction are introduced to evaluate differential tissue response. The safety and constraint enforcement module 210 ensures that probing remains within anatomically safe bounds during such exploration.

Mechanical Assistive and Life-Support Interventions—in some embodiments, the adaptive control architecture is applied to mechanical assistive or life-support interventions requiring controlled mechanical interaction, such as automated cardiopulmonary resuscitation (CPR) or other rhythmic mechanical compression therapies. Sensing elements can include chest wall displacement sensors, compression force sensors, accelerometers, environmental context sensors, and physiological monitors configured to measure parameters indicative of circulatory response. Patient-state inference can estimate patient-specific characteristics including chest wall compliance, skeletal fragility, thoracic stiffness, and effectiveness of mechanical intervention based on measured displacement-force relationships and physiological response metrics. Adaptive control maps inferred compliance and response characteristics to an operational adaptation space defining permissible compression depth, compression frequency, force vector orientation, and actuation timing. Compression parameters may be dynamically modified to optimize circulatory efficiency relative to the specific patient's anatomy. In this embodiment, safety monitoring operates concurrently to ensure that compression depth, applied force, and mechanical repetition rates remain within anatomically safe limits, thereby preventing secondary injury while permitting adaptive optimization within defined safety envelopes.

Adaptive Robotic Physical Therapy—in some embodiments, the disclosed architecture is applied to robotic rehabilitation or physical therapy systems configured to provide adaptive mechanical assistance during patient movement. Sensing elements can include wearable inertial measurement units (IMUs), electromyography (EMG) sensors, joint torque sensors, motion capture systems, and environmental sensors providing continuous data regarding patient movement, muscular activation, and biomechanical state. Patient-state inference can infer muscle fatigue, spasticity, voluntary movement intent, joint stability, coordination quality, or motor recovery progression using sensor-derived biomechanical and physiological features. Adaptive control modifies the operational adaptation space by dynamically adjusting assistance levels (e.g., percentage of actuation force supplied by the robotic device), permissible joint workspace constraints, resistance levels, trajectory shaping, or compliance characteristics. As therapy progresses and patient state evolves (e.g., increasing fatigue or improved voluntary control), the system iteratively updates inferred patient-state representations and modifies control parameters in real time without interruption of the therapy session. Adaptive modification occurs within safety constraints enforced by the system.

Across these embodiments, the underlying control paradigm remains consistent: real-time sensing generates patient-specific data; structured inference produces a contextual state representation; reference-guided evaluation determines whether an acceptance criterion has been satisfied; and adaptive control modifies one or more controllable physical or machine-operational variables within a bounded operational adaptation space. The architecture is therefore device-agnostic and modality-agnostic, while preserving safety constraints, regulatory compatibility, and compatibility with human supervision.

The systems and methods described herein can be implemented using one or more computing devices configured to execute computer-readable instructions stored in one or more memory devices. Each computing device can include one or more processors, such as general-purpose processors, microprocessors, digital signal processors (DSPs), graphics processing units (GPUs), tensor processing units (TPUs), field-programmable gate arrays (FPGAs), application-specific integrated circuits (ASICs), or combinations thereof.

Memory devices include volatile and non-volatile memory, including random-access memory (RAM), read-only memory (ROM), flash memory, solid-state storage, magnetic storage, optical storage, or combinations thereof. Computer-readable instructions stored in such memory devices implement one or more of the procedural protocol execution logic, authorization evaluation logic, autonomy control logic, safety monitoring logic, sensor data processing, artificial intelligence model execution, and human interface functions described herein.

The computing devices are operatively coupled to one or more sensors, robotic actuators, medical devices, or patient support structures via wired or wireless interfaces. Such interfaces include, without limitation, serial interfaces, parallel interfaces, USB, Ethernet, fieldbus protocols, industrial communication protocols, or medical device communication standards. Sensor data is acquired synchronously or asynchronously and is buffered, timestamped, filtered, or otherwise processed prior to use.

In some embodiments, one or more computing devices are communicatively coupled via a communication network to additional computing resources, including remote computing devices, cloud computing environments, or distributed computing platforms. The communication network includes a local area network (LAN), wide area network (WAN), cellular network, private network, virtual private network (VPN), or the Internet. Network communication supports transmission of sensor data, control signals, procedural parameters, artificial intelligence model data, reference databases, and human supervisory input.

Software components implementing the disclosed functionality are organized as one or more modules, services, processes, threads, containers, or virtual machines. In some embodiments, software components are deployed using containerization, orchestration, or distributed execution frameworks. Software components execute on a single computing device or are distributed across multiple computing devices, including edge devices and cloud-based systems.

Artificial intelligence and machine learning models described herein are trained offline, online, or using a combination thereof, and are executed locally, remotely, or in a hybrid configuration. Model execution includes inference, confidence estimation, quality assessment, and generation of candidate actions. Model outputs are combined with rule-based logic, protocol constraints, and human supervisory input prior to controlling physical interaction.

Human supervisory interfaces include graphical user interfaces, touchscreen interfaces, audio interfaces, haptic interfaces, or combinations thereof, and are provided on local or remote computing devices. Such interfaces support visualization of sensor data, procedural state, alerts, candidate actions, and authorization requests, and receive human input affecting system operation.

The disclosed systems and methods are implemented in software, firmware, hardware, or combinations thereof. Certain functions are implemented using dedicated hardware components, while other functions are implemented using software executed by programmable processors. Allocation of functionality between hardware and software varies by embodiment.

The described computing and implementation environment is exemplary, and variations in hardware architecture, networking configuration, software deployment, and execution environment are employed without departing from the scope of the disclosed subject matter.

As used herein, the terms “comprise,” “include,” and plural forms thereof are open-ended and include the listed elements as well as additional elements not expressly listed. The term “and/or” is open-ended and includes one or more of the listed elements and combinations thereof.

The embodiments described are illustrative and not restrictive. Variations and modifications can be made without departing from the spirit or scope of the disclosure, as will be understood by those skilled in the art.

What is claimed is:

1. A system for patient-specific adaptive control of a medical device configured to physically interact with a patient during execution of a medical procedure, the system comprising:
one or more sensing elements configured to acquire sensing data during interaction between the medical device and the patient, and
a computing device operatively coupled to the one or more sensing elements, wherein the computing device is configured to:
generate a structured representation of patient-specific state based on the sensing data;
identify a patient-specific region of interest associated with a procedural objective;
compare procedural data acquired within the region of interest to one or more reference representations and determine whether an acceptance criterion is satisfied;
map the structured representation of patient-specific state to a bounded operational adaptation space defining permissible modifications of controllable device variables; and
generate control signals that modify at least one physical operational state of the medical device within the bounded operational adaptation space in response to determining whether the acceptance criterion is satisfied,
wherein the medical device comprises one or more actuators configured to execute the generated control signals.

2. The system of claim 1, wherein the structured representation of patient-specific state is generated using multi-stage inference comprising: (i) estimation of external body configuration or surface landmarks; (ii) estimation of device-patient interaction state; and (iii) identification or refinement of the patient-specific region of interest.

3. The system of claim 2, wherein estimation of the device-patient interaction state comprises determining at least one of: spatial alignment between the medical device and internal anatomy, contact force distribution, impedance characteristics, tissue compliance, acoustic coupling quality, or a coordinate transform between device space and anatomical space.

4. The system of claim 1, further comprising:
a reference repository operatively coupled to the computing device, the reference repository storing one or more reference representations corresponding to desired anatomical configurations, procedural targets, protocol-defined states, or quality exemplars, and
wherein the computing device retrieves the one or more reference representations based on at least one of an inferred patient-state stage, procedural phase, anatomical target, or identified region of interest.

5. The system of claim 4, wherein the reference representations comprise at least one of geometric templates, anatomical atlases, statistical models, learned feature embeddings, signal profiles, or canonical parameter sets derived from prior procedures.

6. The system of claim 1, wherein the bounded operational adaptation space comprises a multidimensional parameter space defining permissible ranges of at least one controllable device variable selected from: device pose, motion trajectory, actuator force, contact pressure, compliance parameter, acquisition depth, focal configuration, gain setting, scanning trajectory, timing sequence, or energy delivery parameter.

7. The system of claim 6, wherein mapping to the bounded operational adaptation space further comprises dynamically constraining permissible modifications based on at least one of inferred anatomical context, procedural protocol constraints, safety limits, device capability limits, or human supervisory input.

8. The system of claim 1, wherein generation of control signals comprises staged or ordered optimization of multiple controllable device variables.

9. The system of claim 1, wherein the computing device is further configured to independently evaluate proposed modifications within the bounded operational adaptation space against predefined or dynamically determined safety envelopes, and restrict, modify, or override the generated control signals prior to execution by the medical device.

10. The system of claim 1, wherein the computing device, the one or more sensing elements, and the medical device are configured in a closed-loop architecture in which:
(i) execution of the generated control signals modifies the at least one physical operational state of the medical device;
(ii) the modified physical operational state of the medical device produces updated sensing data; and
(iii) the structured representation of patient-specific state is iteratively updated based on the updated sensing data.

11. The system of claim 1, wherein the computing device is further configured to receive human-generated control inputs corresponding to desired operation of the medical device, and to condition execution of the human-generated control inputs such that any resulting actuation of the medical device remains within the bounded operational adaptation space.

12. The system of claim 1, wherein generation of control signals comprises executing an ordered optimization sequence including:
(i) first optimizing a mechanical interaction force between the medical device and the patient to satisfy a coupling threshold; and
(ii) subsequently optimizing at least one signal acquisition parameter within the patient-specific region of interest after the coupling threshold is satisfied.

13. The system of claim 1, wherein the computing device is further configured to execute a structured perturbation routine in which the medical device performs a programmed sequence of incremental physical movements or parameter adjustments to generate sensing data for refining the structured representation of patient-specific state.

14. The system of claim 1, wherein the computing device is further configured to perform a dynamic coordinate transform between a device-centric coordinate system and an anatomical-centric coordinate system defined by an inferred three-dimensional body mesh and one or more identified anatomical landmarks.

15. A method of patient-specific adaptive control of a medical device configured to physically interact with a patient during execution of a medical procedure, the method comprising:
acquiring, using one or more sensing elements, sensing data during interaction between the medical device and the patient;
generating, by a computing device coupled to the one or more sensing elements, a structured representation of patient-specific state based on the sensing data;
identifying, by the computing device, a patient-specific region of interest associated with a procedural objective;
comparing, by the computing device, procedural data acquired within the region of interest to one or more reference representations and determining whether an acceptance criterion is satisfied;
mapping, by the computing device, the structured representation of patient-specific state to a bounded operational adaptation space defining permissible modifications of controllable device variables; and
generating, by the computing device, control signals that modify at least one physical operational state of the medical device within the bounded operational adaptation space in response to determining whether the acceptance criterion is satisfied,
wherein the medical device comprises one or more actuators configured to execute the generated control signals.

16. The method of claim 15, wherein the structured representation of patient-specific state is generated using multi-stage inference comprising: (i) estimation of external body configuration or surface landmarks; (ii) estimation of device-patient interaction state; and (iii) identification or refinement of the patient-specific region of interest.

17. The method of claim 16, wherein estimation of the device-patient interaction state comprises determining at least one of: spatial alignment between the medical device and internal anatomy, contact force distribution, impedance characteristics, tissue compliance, acoustic coupling quality, or a coordinate transform between device space and anatomical space.

18. The method of claim 15, further comprising:
storing, in a reference repository operatively coupled to the computing device, one or more reference representations corresponding to desired anatomical configurations, procedural targets, protocol-defined states, or quality exemplars; and
retrieving, by the computing device, the one or more reference representations based on at least one of an inferred patient-state stage, procedural phase, anatomical target, or identified region of interest.

19. The method of claim 18, wherein the reference representations comprise at least one of geometric templates, anatomical atlases, statistical models, learned feature embeddings, signal profiles, or canonical parameter sets derived from prior procedures.

20. The method of claim 15, wherein the bounded operational adaptation space comprises a multidimensional parameter space defining permissible ranges of at least one controllable device variable selected from: device pose, motion trajectory, actuator force, contact pressure, compliance parameter, acquisition depth, focal configuration, gain setting, scanning trajectory, timing sequence, or energy delivery parameter.

21. The method of claim 20, wherein mapping to the bounded operational adaptation space further comprises dynamically constraining permissible modifications based on at least one of inferred anatomical context, procedural protocol constraints, safety limits, device capability limits, or human supervisory input.

22. The method of claim 15, wherein generation of control signals comprises staged or ordered optimization of multiple controllable device variables.

23. The method of claim 15, further comprising independently evaluating, by the computing device, proposed modifications within the bounded operational adaptation space against predefined or dynamically determined safety envelopes, and restrict, modify, or override the generated control signals prior to execution by the medical device.

24. The method of claim 15, wherein the computing device, the one or more sensing elements, and the medical device are configured in a closed-loop architecture in which:
(i) execution of the generated control signals modifies the at least one physical operational state of the medical device;
(ii) the modified physical operational state of the medical device produces updated sensing data; and
(iii) the structured representation of patient-specific state is iteratively updated based on the updated sensing data.

25. The method of claim 15, wherein the computing device is further configured to receive human-generated control inputs corresponding to desired operation of the medical device, and to condition execution of the human-generated control inputs such that any resulting actuation of the medical device remains within the bounded operational adaptation space.

26. The method of claim 15, wherein generation of control signals comprises executing an ordered optimization sequence including:
(i) first optimizing a mechanical interaction force between the medical device and the patient to satisfy a coupling threshold; and
(ii) subsequently optimizing at least one signal acquisition parameter within the patient-specific region of interest after the coupling threshold is satisfied.

27. The method of claim 15, wherein the computing device is further configured to execute a structured perturbation routine in which the medical device performs a programmed sequence of incremental physical movements or parameter adjustments to generate sensing data for refining the structured representation of patient-specific state.

28. The method of claim 15, wherein the computing device is further configured to perform a dynamic coordinate transform between a device-centric coordinate system and an anatomical-centric coordinate system defined by an inferred three-dimensional body mesh and one or more identified anatomical landmarks.
